(19) United States
(12) Patent Application Publication
(10) Pub. No.: US 2010/0216127 A1
(43) Pub. Date: Aug. 26, 2010

(54) PRIMER SET FOR USE IN DETECTION OF YEAST OF GENUS SACCHAROMYCES

(75) Inventors: Nobuyuki Hayashi, Tokyo-to (JP); Satoshi Yoshida, Tokyo-to (JP); Keiko Kanai, Tokyo-to (JP); Shigehito Ikushima, Tokyo-to (JP); Toshiko Minato, Tokyo-to (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: KIRIN BEER KABUSHIKI KAISHA, TOKYO-TO (JP)

(21) Appl. No.: 12/444,448

(22) PCT Filed: May 17, 2007
(86) PCT No.: PCT/JP2007/060154

§ 371 Date: Apr. 6, 2009

(30) Foreign Application Priority Data
Oct. 5, 2006 (JP) 2006 274166
Dec. 6, 2006 (JP) 2006 329377

(51) Int. Cl. C12Q 1/68; C07H 21/04

(52) U.S. Cl. 435/6; 536/24.32

(57) ABSTRACT

An object of the present invention is to provide a primer set, which can accurately, rapidly and simply identify yeast species of genus Saccharomyces. According to the present invention, there is provided a primer set for use in the detection of the yeast species of genus Saccharomyces, which comprises primers selected from the group consisting of the polynucleotides having the base sequences of SEQ ID NOS: 1 to 17 or 23 to 30, or the homologous polynucleotides thereof.

### TECHNICAL FIELD

The present invention relates to a primer set for use in the detection of a yeast of genus Saccharomyces, and more specifically to a LAMP primer set and a PCR primer set which are for use in the detection of the yeast of genus Saccharomyces. Moreover, the present invention also relates to a method for detecting and quantifying the yeast of genus Saccharomyces, using such primer set.

### BACKGROUND ART

A yeast of genus Saccharomyces has widely been used in the production of bread, and also in the production of alcoholic beverages such as beer, wine, Japanese sake, distilled spirits and whisky. Saccharomyces cerevisiae has been used in the production of alcoholic beverages made by fermentation including top-fermented beer such as ale, wine, Japanese sake, and fruit wine such as cider, and also in the production of distilled liquor such as distilled spirits and whisky. Saccharomyces bayanus has been used in the production of wine, sherry, sparkling wine, etc. A bottom-fermenting yeast used in the production of pilsner beer has been currently classified into Saccharomyces pastorianus (Kurtzman, C. P. & Fell, J. W. The Yeasts, A Taxonomic Study, 4^{th} edition, 1998, Elsevier Science, B. V., The Netherlands, Back, W.: Farbatlas und Handbuch der Geraenkebiologie, Teil I, 1994, Verlag Hans Carl. Nuernberg, Barnett, J. A. et al.: Yeasts, characteristics and identification, 3^{rd} edition, 2000, Cambridge University Press, UK, Seishu Kobo/Koji Kenkyukai (Study Group for Sake Yeasts and Rice Malts): “Studies of Sake Yeasts,” 2003, Shinnihon Printing Inc., Tokyo). Thus, in order to grasp whether or not a yeast used in the production of foods and beverages is a suitable yeast, a technique of identifying the strain type of the yeast of genus Saccharomyces is important.

However, when such yeasts remain in the filtrated alcoholic beverage products or they get mixed from outside, excessive fermentation occurs and causes cloudiness, and it also causes unique odor. Thus, such excessive fermentation further causes pungent bitter taste, so that it affects the quality of products (Back, W.: Farbatlas und Handbuch der Geraenkebiologie, Teil I, 1994, Verlag Hans Carl, Nuernberg, European Brewery Convention: ANALYTICA-MICROBIOLOGICA-EBC, 2^{nd} ed. 2005 Fachverlag Hans Carl, Nuernberg).

Moreover, such yeast of genus Saccharomyces may be isolated also from soft drinks, and in particular from fruit juice drinks. If such yeast gets mixed in products, carbonic acid gas, ethanol and unpleasant odor are generated from sugars such as glucose or saccharose, and the quality of the products are thereby significantly impaired (Back, W.: Farbatlas und Handbuch der Geraenkebiologie, Teil II, 1999, Verlag Hans Carl, Nuernberg).

Thus, if the yeast of genus Saccharomyces proliferates in products, it significantly affects the industry. Accordingly, a technique of rapidly detecting and/or identifying such yeast is important for the control of quality. Moreover, when the yeast of genus Saccharomyces isolated from products is a yeast that has been used during the production process, it is considered that it is caused by leakage from upstream of the production process, an unfavorable filtration step, etc. If the yeast isolated from products gets mixed from outside, it is considered that it is caused by the insufficient washing of a filler, the accumulation of dust in a tube, etc. Accordingly, a technique of identifying yeast isolated from products when contamination is found is important to clarify problems to be solved.

However, taxonomically, Saccharomyces pastorianus, Saccharomyces cerevisiae and Saccharomyces bayanus are extremely closely related. Together with several other types of yeasts such as Saccharomyces paradoxus and Saccharomyces mikatae, they form a taxonomic group named as Saccharomyces sensu stricto (Naumov., G. I. et al., Int. J. Syst. Evol. Microbiol., 2000, vol. 50, 1931-1942). Further, Saccharomyces pastorianus has been considered to be a species formed by the crossing of Saccharomyces cerevisiae with Saccharomyces bayanus, and thus it has been confirmed that Saccharomyces pastorianus is a hybrid of the two above yeast species at a gene level and at a chromosome level (Kielland-Brandt, M. C. et al.: Genetics of brewing yeast. The Yeast, 2^{nd} edn, vol. 6, pp. 223-254, Edited by Wheals, et al., Academic Press, New York, Ryu, S.-L. et al.: Yeast, 1996, vol. 12, 757, Tamai, Y. et al.: Yeast, 1998, vol. 14, 923-933, Tamai, Y. et al.: Yeast, 2000, vol. 16, 1335-1343). As traditional yeast identification methods, morphologic, physiological and biochemical means have been used. In particular, the assimilating ability and fermentative ability of various sugars have been examined in many cases. However, since the phenotypes of strains belonging to Saccharomyces sensu stricto are similar to one another, it is difficult for such traditional methods to distinguish the strains from one another (Naumova, E. S. et al.: Antonievan Leeuwenhoek, 2003, vol. 83, 155-166). As molecular biological approaches for distinguish such strains, there have been known PCR finger printing, DNA/DNA recombination kinetics, karyotype analysis, restriction enzyme cleavage analysis of mitochondrial DNA, analysis of rRNA gene base sequence, rDNA restriction enzyme cleavage analysis, UP-PCR, isozyme analysis, PCR-temperature gradient gel electrophoresis, real time PCR, etc.

To date, a method, which comprises amplifying a portion of the FLO1 gene of a yeast of genus Saccharomyces by a PCR method, or amplifying a portion of an rRNA gene by the PCR method and then identifying whether it is the yeast of genus Saccharomyces, a yeast of another type of genus, a yeast for use in fermentation or a yeast for use in purposes other than fermentation by RFLP, has been developed (Japanese Patent Laid-Open Publication No. 11-56366). Moreover, based on the findings that there are two types of sequences of spacer regions between the 26S rRNA gene and 5S rRNA gene of the bottom-fermenting yeast, PCR primer sets specific for the two types of sequences have been developed (Japanese Patent Laid-Open Publication No. 2001-8684). Furthermore, primers specific for an Lg-FLO1 congenic gene, wherein the N-terminal portion of Lg-FLO1 is ligated to the gene of yeast chromosome IX, have been developed (Japanese Patent Laid-Open Publication No. 2002-233382).

However, since PCR or real-time PCR require high-level temperature control and fluorescence observation, these methods need expensive apparatuses. In addition, after completion of the reaction, the PCR method requires electrophoresis, staining, photography, etc., and thus this method requires a long period of time, until the results are obtained after a gene amplification process. Further, RAPD PCR, the restriction enzyme treatment of an amplification product, the analysis of a base sequence, isozyme analysis, temperature-gradient gel electrophoresis, etc. require a longer period of time and more complicated operations than those of the common PCR, and thus these methods have been problematic when they have been carried out in daily microorganism tests.

On the other hand, Saccharomyces pastorianus has both subgenome derived from Saccharomyces cerevisiae (Sc type) and subgenome derived from Saccharomyces bayanus (Lg type). According to the recent studies, it has been reported that, in the case of the bottom-fermenting yeast, a part of the right arm of Sc-type chromosome XVI is not present, a part of the right arm of Lg-type chromosome III is not present, and a part of the left arm of Lg-type chromosome VII is not present (Naoyuki Umemoto et al., “Production of physical map of beer yeasts and comparative genomic science,” 24^{th} Annual Meeting of the Molecular Biology Society of Japan (2001); Nakao et al., Proceedings of the 29^{th} EBC Congress (2003); Yoshihiro Nakao: Chemistry and Organisms, 2005, vol. 43, No. 9, 559-561; and Japanese Patent Laid-Open Publication No. 2004-283169). However, under the present circumstances, detailed information regarding the chromosomal translocation of Saccharomyces pastorianus has not yet been obtained.

Still further, a LAMP (loop mediated isothermal amplification) method primer set for use in the detection of Saccharomyces pastorianus has been developed (WO2005/093059). However, there has still been room for improvement in detection accuracy.

### SUMMARY OF THE INVENTION

The present inventors have identified the positions of chromosomal translocations of the chromosome XVI right arm, chromosome III right arm and chromosome VII left arm of Saccharomyces pastorianus, and have also analyzed genome around such translocation positions. Moreover, based on such information, the present inventors have succeeded in developing a primer set capable of accurately detecting a yeast of genus Saccharomyces.

Hereinafter, an invention relating to the chromosomal translocation of the chromosome XVI right arm is referred to as first and second embodiments, an invention relating to the chromosomal translocation of the chromosome III right arm is referred to as a third embodiment, and an invention relating to the chromosomal translocation of the chromosome VII left arm is referred to as a fourth embodiment.

### First Embodiment

The present inventors have conducted the genomic analysis of a bottom-fermenting yeast belonging to Saccharomyces pastorianus, and as a result, they have found that the Sc-type chromosome XVI of the bottom-fermenting yeast is translocated with Lg-type chromosome in the ORF of GPH1 of the right arm, and further that it is translocated again in the ORF of QCR2 in the right arm terminal direction, so that it returns to the Sc type. That is, the present inventors have found that only an Lg-type base sequence in the bottom-fermenting yeast exists in a region flanked with the GPH1 and QCR2 of the right arm of the chromosome XVI.

The present inventors have designed a LAMP primer set for use in the detection of Saccharomyces pastorianus based on the sequence (SEQ ID NO: 6) of an Lg-type MET16 gene existing in the region flanked with GPH1 and QCR2, and they have found that, using the thus designed primer set, Saccharomyces pastorianus can be accurately detected. The sequence of the Lg-type MET16 gene is a novel sequence that has not been disclosed in any public database so far.

Specifically, according to the first embodiment of the present invention, there is provided a probe or primer for use in the detection of Saccharomyces pastorianus, which consists of a polynucleotide consisting of at least 10 bases, which hybridizes with a polynucleotide having the base sequence of SEQ ID NO: 6, or a polynucleotide consisting of at least 10 bases, which hybridizes with a polynucleotide having a sequence complementary to the base sequence of SEQ ID NO: 6.

According to the first embodiment of the present invention, there is also provided a LAMP primer set for use in the detection of Saccharomyces pastorianus, which consists of two or more types of the aforementioned primers.

According to the first embodiment of the present invention, there is also provided a PCR primer set for use in the detection of Saccharomyces pastorianus, which consists of two or more types of the aforementioned primers.

According to the first embodiment of the present invention, there is preferably provided a LAMP primer set for use in the detection of Saccharomyces pastorianus, which comprises the following polynucleotides:

a polynucleotide (FIP) having the base sequence of SEQ ID NO: 1, or a polynucleotide consisting of at least 10 bases, which hybridizes with a polynucleotide having a sequence complementary to the base sequence;

a polynucleotide (F3) having the base sequence of SEQ ID NO: 2, or a polynucleotide consisting of at least 10 bases, which hybridizes with a polynucleotide having a sequence complementary to the base sequence;

a polynucleotide (BIP) having the base sequence of SEQ ID NO: 3, or a polynucleotide consisting of at least 10 bases, which hybridizes with a polynucleotide having a sequence complementary to the base sequence; and

a polynucleotide (B3) having the base sequence of SEQ ID NO: 4, or a polynucleotide consisting of at least 10 bases, which hybridizes with a polynucleotide having a sequence complementary to the base sequence.

The present inventors have designed a PCR primer set for use in the detection of Saccharomyces pastorianus based on the chromosomal translocation position of the right arm of chromosome XVI of a bottom-fermenting yeast, and have then found that Saccharomyces pastorianus can be accurately detected using the primer set.

Specifically, according to the first embodiment of the present invention, there is provided a PCR primer set for use in the detection of Saccharomyces pastorianus, which comprises: a polynucleotide having the base sequence of SEQ ID NO: 27, or a polynucleotide consisting of at least 10 bases, which hybridizes with a polynucleotide having a sequence complementary to the base sequence; and a polynucleotide having the base sequence of SEQ ID NO: 28, or a polynucleotide consisting of at least 10 bases, which hybridizes with a polynucleotide having a sequence complementary to the base sequence.

According to the first embodiment of the present invention, there is also provided a PCR primer set for use in the detection of Saccharomyces pastorianus, which comprises: a polynucleotide having the base sequence of SEQ ID NO: 29, or a polynucleotide consisting of at least 10 bases, which hybridizes with a polynucleotide having a sequence complementary to the base sequence; and a polynucleotide having the base sequence of SEQ ID NO: 30, or a polynucleotide consisting of at least 10 bases, which hybridizes with a polynucleotide having a sequence complementary to the base sequence.

According to the first embodiment of the present invention, there is also provided a PCR primer set for use in the detection of Saccharomyces pastorianus, wherein one primer is a polynucleotide consisting of at least 10 bases, which hybridizes with a polynucleotide having the base sequence of SEQ ID NO: 6, or a polynucleotide consisting of at least 10 bases, which hybridizes with a polynucleotide having a sequence complementary to the base sequence of SEQ ID NO: 6, and the other primer is a polynucleotide consisting of at least 10 bases, which hybridizes with a Sc-type base sequence that is out of a region flanked with GPH1 and QCR2 of the right arm of chromosome XVI of a bottom-fermenting yeast, or a sequence complementary thereto.

According to the first embodiment of the present invention, there is provided a method for detecting Saccharomyces pastorianus, which comprises performing a nucleic acid amplification reaction by a LAMP method using the LAMP primer set of the first embodiment.

According to the first embodiment of the present invention, there is also provided a method for detecting Saccharomyces pastorianus, which comprises performing a nucleic acid amplification reaction by a PCR method using the PCR primer set of the first embodiment.

According to the first embodiment of the present invention, there is further provided a method for detecting Saccharomyces pastorianus, which comprises detecting a hybridization complex using the probe of the first embodiment.

### Second Embodiment

The present inventors have found that only an Lg-type base sequence in the bottom-fermenting yeast exists in a region flanked with GPH1 and QCR2 of the right arm of chromosome XVI. That is, it was revealed that the Sc-type base sequence in the region flanked with GPH1 and QCR2 of the right arm of chromosome XVI is not present in Saccharomyces pastorianus or Saccharomyces bayanus, and thus that the aforementioned Sc-type base sequence is specific for Saccharomyces cerevisiae.

The present inventors have designed a LAMP primer set for use in the detection of Saccharomyces cerevisiae based on the sequence of a Sc-type MET16 gene existing in the region flanked with GPH1 and QCR2. The inventors have then found that Saccharomyces cerevisiae can be accurately detected using the primer set.

Specifically, according to the second embodiment of the present invention, there is provided a LAMP primer set for use in the detection of Saccharomyces cerevisiae, which comprises the following polynucleotides:

a polynucleotide (FIP) having the base sequence of SEQ ID NO: 7, or a polynucleotide consisting of at least 10 bases, which hybridizes with a polynucleotide having a sequence complementary to the base sequence;

a polynucleotide (F3) having the base sequence of SEQ ID NO: 8, or a polynucleotide consisting of at least 10 bases, which hybridizes with a polynucleotide having a sequence complementary to the base sequence;

a polynucleotide (BIP) having the base sequence of SEQ ID NO: 9, or a polynucleotide consisting of at least 10 bases, which hybridizes with a polynucleotide having a sequence complementary to the base sequence; and

a polynucleotide (B3) having the base sequence of SEQ ID NO: 10, or a polynucleotide consisting of at least 10 bases, which hybridizes with a polynucleotide having a sequence complementary to the base sequence.

According to the second embodiment of the present invention, there is provided a method for detecting Saccharomyces cerevisiae, which comprises performing a nucleic acid amplification reaction by a LAMP method using the LAMP primer set of the second embodiment.

### Third Embodiment

The present inventors have found that the Lg-type chromosome III of the bottom-fermenting yeast is translocated with Sc-type chromosome at the MAT locus of the right arm, and that only a Sc-type base sequence exists from the MAT locus of the chromosome III right arm to the terminus thereof in the bottom-fermenting yeast. That is, it was revealed that, since an Lg-type base sequence is not present from the MAT locus of the chromosome III right arm to the terminus thereof in Saccharomyces cerevisiae or Saccharomyces pastorianus, the base sequence of Saccharomyces bayanus corresponding to that region is specific for Saccharomyces bayanus.

The present inventors have designed a LAMP primer set for use in the detection of Saccharomyces bayanus based on the sequence of a RAD18 homologous gene existing in a region sandwiched between the MAT locus and the terminus. The inventors have then found that Saccharomyces bayanus can be accurately detected using the primer set.

Specifically, according to the third embodiment of the present invention, there is provided a LAMP primer set for use in the detection of Saccharomyces bayanus, which comprises the following polynucleotides:

a polynucleotide (FIP) having the base sequence of SEQ ID NO: 13, or a polynucleotide consisting of at least 10 bases, which hybridizes with a polynucleotide having a sequence complementary to the base sequence;

a polynucleotide (F3) having the base sequence of SEQ ID NO: 14, or a polynucleotide consisting of at least 10 bases, which hybridizes with a polynucleotide having a sequence complementary to the base sequence;

a polynucleotide (BIP) having the base sequence of SEQ ID NO: 15, or a polynucleotide consisting of at least 10 bases, which hybridizes with a polynucleotide having a sequence complementary to the base sequence; and

a polynucleotide (B3) having the base sequence of SEQ ID NO: 16, or a polynucleotide consisting of at least 10 bases, which hybridizes with a polynucleotide having a sequence complementary to the base sequence.

The present inventors have also designed a PCR primer set for use in the detection of Saccharomyces pastorianus based on the chromosomal translocation position of the right arm of chromosome III of a bottom-fermenting yeast. The inventors have then found that Saccharomyces pastorianus can be accurately detected using the primer set.

Specifically, according to the third embodiment of the present invention, there is provided a PCR primer set for use in the detection of Saccharomyces pastorianus, which comprises: a polynucleotide having the base sequence of SEQ ID NO: 23, or a polynucleotide consisting of at least 10 bases, which hybridizes with a polynucleotide having a sequence complementary to the base sequence; and a polynucleotide having the base sequence of SEQ ID NO: 24, or a polynucleotide consisting of at least 10 bases, which hybridizes with a polynucleotide having a sequence complementary to the base sequence.

According to the third embodiment of the present invention, there is provided a method for detecting Saccharomyces bayanus, which comprises performing a nucleic acid amplification reaction by a LAMP method using the LAMP primer set of the third embodiment.

According to the third embodiment of the present invention, there is also provided a method for detecting Saccharomyces pastorianus, which comprises performing a nucleic acid amplification reaction by a PCR method using the PCR primer set of the third embodiment.

### Fourth Embodiment

The present inventors have found that the Lg-type chromosome VII of the bottom-fermenting yeast is translocated with Sc-type chromosome in the ORF of a left arm KEM1 gene. That is, the inventors have found that only a Sc-type base sequence exists from the KEM1 locus of the chromosome VII left arm to the terminus of the left arm in the bottom-fermenting yeast.

The present inventors have also designed a PCR primer set for use in the detection of Saccharomyces pastorianus based on the chromosomal translocation position of the chromosome VII left arm of the bottom-fermenting yeast. The inventors have then found that Saccharomyces pastorianus can be accurately detected using the primer set.

Specifically, according to the fourth embodiment of the present invention, there is provided a PCR primer set for use in the detection of Saccharomyces pastorianus, which comprises: a polynucleotide having the base sequence of SEQ ID NO: 25, or a polynucleotide consisting of at least 10 bases, which hybridizes with a polynucleotide having a sequence complementary to the base sequence; and a polynucleotide having the base sequence of SEQ ID NO: 26, or a polynucleotide consisting of at least 10 bases, which hybridizes with a polynucleotide having a sequence complementary to the base sequence.

According to the fourth embodiment of the present invention, there is provided a method for detecting Saccharomyces pastorianus, which comprises performing a nucleic acid amplification reaction by a PCR method using the PCR primer set of the fourth embodiment.

According to the primer sets according to the present invention, the yeast of genus Saccharomyces can be accurately detected at a species level. In particular, the LAMP primer sets according to the present invention can be used in a nucleic acid amplification reaction by a LAMP method, and in a detection of a target species based on the presence or absence of an amplified product. Thus, according to the LAMP primer sets of the present invention, the yeast of genus Saccharomyces can be accurately, rapidly and simply identified at a species level.

According to the LAMP primer sets according to the present invention, the number of cells contained in a sample can also be measured. Thus, according to the LAMP primer sets according to the present invention, Saccharomyces pastorianus, Saccharomyces cerevisiae, and Saccharomyces bayanus can be accurately quantified.

The yeasts of genus Saccharomyces are yeast species that cloud various types of beverages such as alcoholic beverages and soft drinks. Thus, the presence or absence of these yeast species may be used as an indicator of the quality control of various types of beverages. Accordingly, the primer sets according to the present invention are useful for the quality control of various types of beverages (for example, alcoholic beverages and soft drinks, particularly, beer, low-malt beer (happoshu), and wine) and the examination of environmental samples.

### BRIEF DESCRIPTION OF THE DRAWINGS

FIG. 1 shows the reaction specificity of a primer set (LGM1LB1) for use in the detection of Saccharomyces pastorianus for detection target species. The following strains were used: Saccharomyces cerevisiae NBRC10217, Saccharomyces bayanus NBRC11022, Saccharomyces pastorianus NBRC11024, NBRC11023 and NBRC10610, Saccharomyces cerevisiae var. diastaticus DSM70487, Saccharomyces paradoxus NBRC10609, Saccharomyces cariocanus NBRC10947, Saccharomyces mikatae NBRC1815, Saccharomyces kudriavzevii NBRC 1802, Saccharomyces exiguous NBRC1128, Saccharomyces servazzii NBRC1838, Saccharomyces unisporus NBRC0316, Saccharomyces dairenensis NBRC0211, Saccharomyces kluyveri NBRC1685, Nega: no genomic DNA added.

FIG. 2 shows an approximate curve formed by the colony formation number of Saccharomyces pastorianus and the detection time by the LAMP method using LGM1LB1. The threshold time on the horizontal axis indicates the reaction time when the turbidity exceeded 0.1.

FIG. 3 shows the reaction specificity of a primer set (SSC1LB1) for use in the detection of genus Saccharomyces for detection target species. The following strains were used: Saccharomyces cerevisiae NBRC10217, Saccharomyces bayanus NBRC11022, Saccharomyces pastorianus NBRC11024, NBRC11023 and NBRC10610, Saccharomyces cerevisiae var. diastaticus DSM70487, Saccharomyces paradoxus NBRC10609, Saccharomyces cariocanus NBRC10947, Saccharomyces mikatae NBRC1815, Saccharomyces kudriavzevii NBRC 1802, Saccharomyces exiguous NBRC1128, Saccharomyces servazzii NBRC1838, Saccharomyces unisporus NBRC0316, Saccharomyces dairenensis NBRC0211, Saccharomyces kluyveri NBRC1685, Nega: no genomic DNA added.

FIG. 4 shows the reaction specificity of a primer set (SBFY1LF1LB1) for use in the detection of a bottom-fermenting yeast for detection target species. The following strains were used: Saccharomyces cerevisiae NBRC10217, Saccharomyces bayanus NBRC11022, Saccharomyces pastorianus NBRC11024, NBRC11023 and NBRC10610, Saccharomyces cerevisiae var. diastaticus DSM70487, Saccharomyces paradoxus NBRC10609, Saccharomyces cariocanus NBRC10947, Saccharomyces mikatae NBRC1815, Saccharomyces kudriavzevii NBRC 1802, Saccharomyces exiguous NBRC1128, Saccharomyces servazzii NBRC1838, Saccharomyces unisporus NBRC0316, Saccharomyces dairenensis NBRC0211, Saccharomyces kluyveri NBRC1685, Nega: no genomic DNA added.

### DETAILED DESCRIPTION OF THE INVENTION

### Primers and Primer Sets

The LAMP primer set according to the present invention consists of 4 types of primers, namely, FIP, F3, BIP and B3. These primers correspond to 6 regions on a target nucleotide sequence. Specifically, regions F3c, F2c, F1c, B1, B2 and B3 are determined in this order from the 3′-terminal side to the 5′-terminal side on the target base sequence. Thereafter, 4 types of primers, namely, FIP, F3, BIP and B3 are produced with respect to the 6 regions. Herein, regions complementary to the regions F3c, F2c and F1c are F3, F2 and F1, respectively. In addition, regions complementary to the regions B1, B2 and B3 are B1c, B2c and B3c, respectively.

FIP is a primer produced in such a way that it has an F2 region complementary to the F2c region of the target sequence on the 3′-terminal side and that it has the same sequence as the F1c region of the target gene on the 5′-terminal side. If necessary, a restriction enzyme site may be introduced into the portion between F1c and F2 of the FIP primer.

F3 is a primer produced in such a way that it has an F3 region complementary to the F3c region of the target gene.

BIP is a primer produced in such a way that it has a B2 region complementary to the B2c region of the target sequence on the 3′-terminal side and that it has the same sequence as the B1c region of the target gene on the 5′-terminal side. If necessary, a restriction enzyme site may be introduced into the portion between B1c and B2 of the BIP primer.

B3 is a primer produced in such a way that it has a B3 region complementary to the B3c region of the target gene.

When restriction enzyme sites are contained in the FIP and BIP primers, an amplified product is treated with restriction enzymes after completion of the nucleic acid amplification reaction by the LAMP method, so that it can be observed that a single band is formed after performing electrophoresis. In this case, if the target sequence contains a restriction enzyme site, it may not be necessary to artificially introduce such a restriction enzyme site into the primers.

When the LAMP primer set according to the present invention is used, one or two types of Loop primers (an LF primer or an LB primer) may be added in order to accelerate the nucleic acid amplification reaction. Such a Loop primer is designed such that it is annealed to a region between F1 and F2 or a region between B1 and B2, and it is then added to the LAMP reaction system. Thus, these primers bind to Loop portions that are not used in the nucleic acid amplification process, so that a nucleic acid reaction can be promoted using all the Loop portions as origins, and so that the nucleic acid amplification reaction can be thereby accelerated (e.g. Japanese Patent Laid-Open Publication No. 2002-345499).

Specifically, among the LAMP primer sets of the first embodiment, the LAMP primer set consisting of polynucleotides having the base sequences of SEQ ID NOS: 1 to 4 or homologous polynucleotides thereof may further comprise, as a Loop primer, a polynucleotide (LB) having the base sequence of SEQ ID NO: 5, or a polynucleotide consisting of at least 10 bases, which hybridizes with a polynucleotide having a sequence complementary to the base sequence.

The LAMP primer set of the second embodiment may further comprise, as a Loop primer(s), any one or both of: a polynucleotide (LF) having the base sequence of SEQ ID NO: 11, or a polynucleotide consisting of at least 10 bases, which hybridizes with a polynucleotide having a sequence complementary to the base sequence; and a polynucleotide (LB) having the base sequence of SEQ ID NO: 12, or a polynucleotide consisting of at least 10 bases, which hybridizes with a polynucleotide having a sequence complementary to the base sequence.

The LAMP primer set of the third embodiment may further comprise, as a Loop primer, a polynucleotide (LB) having the base sequence of SEQ ID NO: 17, or a polynucleotide consisting of at least 10 bases, which hybridizes with a polynucleotide having a sequence complementary to the base sequence.

In the present invention, not only the polynucleotides having the base sequences of SEQ ID NOS: 1 to 5 and 7 to 30, but also polynucleotides hybridizing with polynucleotides having sequences complementary to the base sequences of SEQ ID NOS: 1 to 5 and 7 to 30 (which may also be referred to as “homologous polynucleotides” in the present specification) can be used as primers or probes.

Moreover, in the present invention, a polynucleotide consisting of at least 10 bases hybridizing with a polynucleotide having the base sequence of SEQ ID NO: 6, and a polynucleotide consisting of at least 10 bases hybridizing with a polynucleotide having a sequence complementary to the base sequence of SEQ ID NO: 6, can be used as LAMP primers, PCR primers, and probes.

The term “hybridize” is used in the present specification to mean that a certain polynucleotide hybridizes with a target polynucleotide, but that it does not substantially hybridize with polynucleotides other than the target polynucleotide. Such hybridization can be carried out under stringent conditions. Herein, “stringent conditions” can be determined depending on the Tm(° C.) of a double strand of a primer sequence and a complementary strand thereof, a necessary salt concentration, etc. A technique of selecting a sequence used as a probe and then determining stringent conditions suitable therefor is well known to persons skilled in the art. (Refer to e.g. J. Sambrook, E. F. Frisch, T. Maniatis; Molecular Cloning 2^{nd} edition, Cold Spring Harbor Laboratory (1989), etc.) As such stringent conditions, a hybridization reaction is carried out at a temperature slightly lower than the Tm determined based on a nucleotide sequence (for example, a temperature that is approximately 0° C. to 5° C. lower than the Tm), in a suitable buffer solution commonly used in hybridization. In addition, as other stringent conditions, washing after the hybridization reaction is carried out in a high concentration of low-salt-concentration solution. Examples of such stringent conditions include washing conditions wherein washing is carried out in a 6×SSC/0.05% sodium pyrophosphate solution at temperatures of 37° C. (for an oligonucleotide consisting of approximately 14 bases), 48° C. (for an oligonucleotide consisting of approximately 17 bases), 55° C. (for an oligonucleotide consisting of approximately 20 bases) and 60° C. (for an oligonucleotide consisting of approximately 23 bases).

The nucleotide length of a homologous polynucleotide is at least 10 bases.

In the case of the LAMP primers, the nucleotide length of each of the homologous polynucleotides of FIP and BIP may be preferably at least 30 bases (for example 30 to 60 bases), and more preferably at least 42 bases (for example, 42 to 57 bases).

Moreover, the nucleotide length of each of the homologous polynucleotides of F3, B3, LF and LB may be preferably at least 12 bases (for example, 12 to 30 bases), and more preferably at least 18 bases (for example, 18 to 25 bases and 18 to 30 bases).

In the case of the PCR primers, the nucleotide length of each of the homologous polynucleotides of polynucleotides having the base sequences of SEQ ID NOS: 23 to 30 may be preferably at least 15 bases (for example, 15 to 30 bases), more preferably at least 18 bases (for example, 18 to 24 bases and 18 to 30 bases), and particularly preferably at least 20 bases (for example, 20 to 25 bases and 20 to 30 bases).

Such a homologous polynucleotide may be a polynucleotide comprising at least 10, preferably at least 15, more preferably at least 18, particularly preferably at least 20 contiguous nucleotides of the corresponding base sequence.

Examples of polynucleotides homologous to the polynucleotides having the base sequences of SEQ ID NOS: 1 to 5 and 7 to 30 are as follows.
A homologous polynucleotide of FIP having the base sequence of SEQ ID NO: 1: a polynucleotide comprising at least 42 (42 to 52), and more preferably at least 47 (47 to 52) contiguous nucleotides of SEQ ID NO: 1 (in which one or several mutations may be introduced) (wherein the nucleotide length may be at most 60 bases, and preferably at most 57 bases)
A homologous polynucleotide of F3 having the base sequence of SEQ ID NO: 2: a polynucleotide comprising at least 15 (15 to 19), and more preferably at least 18 (18 or 19) contiguous nucleotides of SEQ ID NO: 2 (in which one or several mutations may be introduced) (wherein the nucleotide length may be at most 30 bases, preferably at most 25 bases, and more preferably at most 21 bases)
A homologous polynucleotide of BIP having the base sequence of SEQ ID NO: 3: a polynucleotide comprising at least 36 (36 to 42), and more preferably at least 38 (38 to 42) contiguous nucleotides of SEQ ID NO: 3 (in which one or several mutations may be introduced) (wherein the nucleotide length may be at most 60 bases, preferably at most 53 bases, and more preferably at most 47 bases)
A homologous polynucleotide of B3 having the base sequence of SEQ ID NO: 4: a polynucleotide comprising at least 19 (19 to 23), and more preferably at least 21 (21 to 23) contiguous nucleotides of SEQ ID NO: 4 (in which one or several mutations may be introduced) (wherein the nucleotide length may be at most 30 bases, and more preferably at most 25 bases)
A homologous polynucleotide of LB having the base sequence of SEQ ID NO: 5: a polynucleotide comprising at least 18 (18 to 22), and more preferably at least 20 (20 to 22) contiguous nucleotides of SEQ ID NO: 5 (in which one or several mutations may be introduced) (wherein the nucleotide length may be at most 30 bases and preferably at most 25 bases)
A homologous polynucleotide of FIP having the base sequence of SEQ ID NO: 7: a polynucleotide comprising at least 38 (38 to 47), and more preferably at least 42 (42 to 47) contiguous nucleotides of SEQ ID NO: 7 (in which one or several mutations may be introduced) (wherein the nucleotide length may be at most 60 bases and preferably at most 53 bases)
A homologous polynucleotide of F3 having the base sequence of SEQ ID NO: 8: a polynucleotide comprising at least 18 (18 to 22), and more preferably at least 19 (19 to 22) contiguous nucleotides of SEQ ID NO: 8 (in which one or several mutations may be introduced) (wherein the nucleotide length may be at most 30 bases, preferably at most 25 bases, and more preferably at most 23 bases)
A homologous polynucleotide of BIP having the base sequence of SEQ ID NO: 9: a polynucleotide comprising at least 42 (42 to 57), more preferably at least 47 (47 to 57), particularly preferably at least 51 (51 to 57), and most preferably at least 53 (53 to 57) contiguous nucleotides of SEQ ID NO: 9 (in which one or several mutations may be introduced) (wherein the nucleotide length may be at most 60 bases)
A homologous polynucleotide of B3 having the base sequence of SEQ ID NO: 10: a polynucleotide comprising at least 19 (19 to 25), and more preferably at least 22 (22 to 25) contiguous nucleotides of SEQ ID NO: 10 (in which one or several mutations may be introduced) (wherein the nucleotide length may be at most 30 bases)
A homologous polynucleotide of LF having the base sequence of SEQ ID NO: 11: a polynucleotide comprising at least 19 (19 to 25), and more preferably at least 22 (22 to 25) contiguous nucleotides of SEQ ID NO: 11 (in which one or several mutations may be introduced) (wherein the nucleotide length may be at most 30 bases)
A homologous polynucleotide of LB having the base sequence of SEQ ID NO: 12: a polynucleotide comprising at least 19 (19 to 25), and more preferably at least 22 (22 to 25) contiguous nucleotides of SEQ ID NO: 12 (in which one or several mutations may be introduced) (wherein the nucleotide length may be at most 30 bases)
A homologous polynucleotide of FIP having the base sequence of SEQ ID NO: 13: a polynucleotide comprising at least 42 (42 to 53), and more preferably at least 48 (48 to 53) contiguous nucleotides of SEQ ID NO: 12 (in which one or several mutations may be introduced) (wherein the nucleotide length may be at most 60 bases and preferably at most 57 bases)
A homologous polynucleotide of F3 having the base sequence of SEQ ID NO: 14: a polynucleotide comprising at least 18 (18 to 21), and more preferably at least 19 (19 to 21) contiguous nucleotides of SEQ ID NO: 13 (in which one or several mutations may be introduced) (wherein the nucleotide length may be at most 30 bases, preferably at most 25 bases, and more preferably at most 23 bases)
A homologous polynucleotide of BIP having the base sequence of SEQ ID NO: 15: a polynucleotide comprising at least 37 (37 to 44), and more preferably at least 42 (42 to 44) contiguous nucleotides of SEQ ID NO: 14 (in which one or several mutations may be introduced) (wherein the nucleotide length may be at most 60 bases, preferably at most 53 bases, and more preferably at most 47 bases)
A homologous polynucleotide of B3 having the base sequence of SEQ ID NO: 16: a polynucleotide comprising at least 19 (19 to 25), and more preferably at least 22 (22 to 25) contiguous nucleotides of SEQ ID NO: 15 (in which one or several mutations may be introduced) (wherein the nucleotide length may be at most 30 bases)
A homologous polynucleotide of LB having the base sequence of SEQ ID NO: 17: a polynucleotide comprising at least 14 (14 to 18), and more preferably at least 16 (16 to 18) contiguous nucleotides of SEQ ID NO: 16 (in which one or several mutations may be introduced) (wherein the nucleotide length may be at most 30 bases, preferably at most 25 bases, and more preferably at most 22 bases)
A homologous polynucleotide of FIP having the base sequence of SEQ ID NO: 18: a polynucleotide comprising at least 38 (38 to 47), and more preferably at least 42 (42 to 47) contiguous nucleotides of SEQ ID NO: 18 (in which one or several mutations may be introduced) (wherein the nucleotide length may be at most 60 bases and preferably at most 53 bases)
A homologous polynucleotide of F3 having the base sequence of SEQ ID NO: 19: a polynucleotide comprising at least 18 (18 to 20), and more preferably at least 19 (19 or 20) contiguous nucleotides of SEQ ID NO: 19 (in which one or several mutations may be introduced) (wherein the nucleotide length may be at most 30 bases, preferably at most 25 bases, and more preferably at most 22 bases)
A homologous polynucleotide of BIP having the base sequence of SEQ ID NO: 20: a polynucleotide comprising at least 36 (36 to 42), and more preferably at least 38 (38 to 42) contiguous nucleotides of SEQ ID NO: 20 (in which one or several mutations may be introduced) (wherein the nucleotide length may be at most 60 bases, preferably at most 53 bases, and more preferably at most 47 bases)
A homologous polynucleotide of B3 having the base sequence of SEQ ID NO: 21: a polynucleotide comprising at least 18 (18 to 20), and more preferably at least 19 (19 or 20) contiguous nucleotides of SEQ ID NO: 21 (in which one or several mutations may be introduced) (wherein the nucleotide length may be at most 30 bases, preferably at most 25 bases, and more preferably at most 22 bases)
A homologous polynucleotide of LB having the base sequence of SEQ ID NO: 22: a polynucleotide comprising at least 18 (18 to 20), and more preferably at least 19 (19 or 20) contiguous nucleotides of SEQ ID NO: 22 (in which one or several mutations may be introduced) (wherein the nucleotide length may be at most 30 bases, preferably at most 25 bases, and more preferably at most 22 bases)
A homologous polynucleotide of a polynucleotide having the base sequence of SEQ ID NO: 23: a polynucleotide comprising at least 19 (19 to 23), and more preferably at least 21 (21 to 23) contiguous nucleotides of SEQ ID NO: 17 (in which one or several mutations may be introduced) (wherein the nucleotide length may be at most 30 bases and preferably at most 25 bases)
A homologous polynucleotide of a polynucleotide having the base sequence of SEQ ID NO: 24: a polynucleotide comprising at least 20 (20 to 24), and more preferably at least 22 (22 to 24) contiguous nucleotides of SEQ ID NO: 18 (in which one or several mutations may be introduced) (wherein the nucleotide length may be at most 30 bases, and preferably at most 25 bases)
A homologous polynucleotide of a polynucleotide having the base sequence of SEQ ID NO: 25: a polynucleotide comprising at least 18 (18 to 21), and more preferably at least 19 (19 to 21) contiguous nucleotides of SEQ ID NO: 19 (in which one or several mutations may be introduced) (wherein the nucleotide length may be at most 30 bases and preferably at most 25 bases)
A homologous polynucleotide of a polynucleotide having the base sequence of SEQ ID NO: 26: a polynucleotide comprising at least 14 (14 to 18), and more preferably at least 16 (16 to 18) contiguous nucleotides of SEQ ID NO: 20 (in which one or several mutations may be introduced) (wherein the nucleotide length may be at most 30 bases, preferably at most 25 bases, and more preferably at most 23 bases)
A homologous polynucleotide of a polynucleotide having the base sequence of SEQ ID NO: 27: a polynucleotide comprising at least 16 (16 to 20), and more preferably at least 18 (18 to 20) contiguous nucleotides of SEQ ID NO: 21 (in which one or several mutations may be introduced) (wherein the nucleotide length may be at most 30 bases, preferably at most 25 bases, and more preferably at most 23 bases)
A homologous polynucleotide of a polynucleotide having the base sequence of SEQ ID NO: 28: a polynucleotide comprising at least 16 (16 to 20), and more preferably at least 18 (18 to 20) contiguous nucleotides of SEQ ID NO: 22 (in which one or several mutations may be introduced) (wherein the nucleotide length may be at most 30 bases, preferably at most 25 bases, and more preferably at most 23 bases)
A homologous polynucleotide of a polynucleotide having the base sequence of SEQ ID NO: 29: a polynucleotide comprising at least 16 (16 to 20), and more preferably at least 18 (18 to 20) contiguous nucleotides of SEQ ID NO: 23 (in which one or several mutations may be introduced) (wherein the nucleotide length may be at most 30 bases, preferably at most 25 bases, and more preferably at most 23 bases)
A homologous polynucleotide of a polynucleotide having the base sequence of SEQ ID NO: 30: a polynucleotide comprising at least 16 (16 to 20), and more preferably at least 18 (18 to 20) contiguous nucleotides of SEQ ID NO: 24 (in which one or several mutations may be introduced) (wherein the nucleotide length may be at most 30 bases, preferably at most 25 bases, and more preferably at most 23 bases)

In the present invention, the polynucleotide consisting of at least 10 bases hybridizing with the polynucleotide having the base sequence of SEQ ID NO: 6, and the polynucleotide consisting of at least 10 bases hybridizing with the polynucleotide having a sequence complementary to the base sequence of SEQ ID NO: 6 may be a polynucleotide comprising at least 10 contiguous nucleotides of a sequence complementary to the base sequence of SEQ ID NO: 6, and a polynucleotide comprising at least 10 contiguous nucleotides of the base sequence of SEQ ID NO: 6, respectively.

When a polynucleotide produced based on the base sequence of SEQ ID NO: 6 is used as a LAMP primer (FIP and BIP), a polynucleotide comprising at least 42 (for example, 42 to 57) contiguous nucleotides (in which one or several mutations may be introduced) of the base sequence of SEQ ID NO: 6 or a sequence complementary thereto (wherein the nucleotide length may be at most 60 bases, and preferably at most 57 bases) can be used as a primer.

When a polynucleotide produced based on the base sequence of SEQ ID NO: 6 is used as a LAMP primer (F3, B3, LB, and LF), a polynucleotide comprising at least 18 (for example, 18 to 25) contiguous nucleotides (in which one or several mutations may be introduced) of the base sequence of SEQ ID NO: 6 or a sequence complementary thereto (wherein the nucleotide length may be at most 30 bases, and preferably at most 25 bases) can be used as a primer.

When a polynucleotide produced based on the base sequence of SEQ ID NO: 6 is used as a PCR primer, a polynucleotide comprising at least 15 (for example, 15 to 30), more preferably at least 18 (for example, 18 to 24 and 18 to 30), and particularly preferably at least 20 (for example, 20 to 25 and 20 to 30) contiguous nucleotides (in which one or several mutations may be introduced) of the base sequence of SEQ ID NO: 6 or a sequence complementary thereto (wherein the nucleotide length may be at most 30 bases, preferably at most 25 bases, and more preferably at most 24 bases) can be used as a primer.

In the present invention, a PCR primer pair for detecting Saccharomyces pastorianus can be selected based on a polynucleotide having the base sequence of SEQ ID NO: 6. Specifically, in the PCR primers can be selected, such that one of two primers makes a pair with the base sequence of SEQ ID NO: 6, that the other primer makes a pair with a sequence complementary to the base sequence of SEQ ID NO: 6, and that the one primer makes a pair with an extending strain elongated by the other primer.

Also, in the present invention, a LAMP primer set for detecting Saccharomyces pastorianus can be selected based on the polynucleotide having the base sequence of SEQ ID NO: 6. Specifically, 4 types of primers necessary for the implementation of the LAMP method, namely, FIP, F3, BIP and B3 are designed as described above, and as necessary, Loop primers such as LF and LB can also be used.

Moreover, each of a homologous polynucleotide and a polynucleotide produced based on the base sequence of SEQ ID NO: 6 may be a polynucleotide having at least 90%, preferably at least 95% identity with each corresponding base sequence. The numerical value of identity can be calculated in accordance with the algorithm well known in the art field. For example, the numerical value of identity can be calculated using BLAST (http://www.ddbj.nig.ac.jp/search/blast-j.html).

Furthermore, such a homologous polynucleotide and a polynucleotide produced based on the base sequence of SEQ ID NO: 6 may be a polynucleotide that consists of a modified base sequence in which one or several mutations are introduced with respect to the corresponding base sequence and hybridizes with a polynucleotide having a sequence complementary to the corresponding base sequence.

Herein, the term “mutation”, which may be the same or different, may be selected from a substitution, a deletion, an insertion and an addition. Such mutation may be preferably selected from “one base substitution” in which a certain base is substituted with another base, “one base deletion” in which a certain base is deleted, “one base insertion” in which a certain base is inserted, and “one base addition” in which a certain base is added. The number of mutations may be 1 to 6 bases, 1, 2, 3 or 4 bases, 1 or 2 bases, or 1 base.

In the present invention, the term “polynucleotide” means to include DNA, RNA and PNA (peptide nucleic acid).

A polynucleotide that constitutes the primer set according to the present invention may be prepared by the chemical synthesis of a nucleic acid according to an ordinary method such as a phosphate triester method (Hunkapiller, M. et al., Nature, 310, 105, 1984). Otherwise, the total DNA of a strain as a detection target may be obtained, and a DNA fragment containing a nucleotide sequence of interest may be then obtained, as appropriate, by the PCR method or the like, based on the nucleotide sequences disclosed in the present specification.

A specific embodiment of the detection method according to the present invention may include a detection method which comprises performing a nucleic acid amplification reaction on a nucleic acid sample by a LAMP method and then detecting the presence or absence of a nucleic acid amplification product. Specifically, there are provided the following methods.

In the first embodiment according to the present invention, there is provided a method for detecting Saccharomyces pastorianus, which comprises:

(a) performing a nucleic acid amplification reaction on a nucleic acid contained in a sample by a LAMP method using the LAMP primer set of the first embodiment according to the present invention; and
(b) detecting the presence or absence of an amplification product,
wherein the generation of the amplification product indicates the presence of Saccharomyces pastorianus.

In the second embodiment according to the present invention, there is provided a method for detecting Saccharomyces cerevisiae, which comprises

(c) performing a nucleic acid amplification reaction on a nucleic acid contained in a sample by a LAMP method using the LAMP primer set of the second embodiment according to the present invention; and
(d) detecting the presence or absence of an amplification product,
wherein the generation of the amplification product indicates the presence of Saccharomyces cerevisiae.

In the third embodiment according to the present invention, there is provided a method for detecting Saccharomyces bayanus, which comprises

(e) performing a nucleic acid amplification reaction on a nucleic acid contained in a sample by a LAMP method using the LAMP primer set of the third embodiment according to the present invention; and
(f) detecting the presence or absence of an amplification product,
wherein the generation of the amplification product indicates the presence of Saccharomyces bayanus.

A sample subjected to the nucleic acid amplification process by the LAMP method may be prepared in such a way that cells contained in the sample are cultured, and a nucleic acid is extracted from the cultured cells, or such a nucleic acid is extracted without such a culture process. The preparation of a nucleic acid sample, such as the culture of cells, the extraction of a nucleic acid will be described later.

In the nucleic acid amplification process by the LAMP method, an amplification reaction is performed on a nucleic acid contained in a sample. Such a nucleic acid amplification reaction by the LAMP method will be described later.

In a case where a detection target species exists in a sample, a specific region as a target is amplified, and an amplification product is generated. When such an amplification product is generated, a sample solution subjected to a nucleic acid amplification reaction becomes clouded. Thus, the presence or absence of the amplification product can be determined by measuring the turbidity of the sample solution. The measurement of the turbidity by the LAMP method is well known. The turbidity can be measured using a commercially available end point turbidity measurement apparatus (e.g. LA-100 manufactured by Teramecs Co., Ltd.) or a real-time turbidity measurement apparatus (e.g. LA-200 manufactured by Teramecs Co., Ltd.).

As described in the examples as given later, a time required until a sample solution reaches a certain turbidity is measured, so as to determine the number of cells contained in a test sample. Specifically, in another aspect of the detection method according to the present invention, there is provided a method for quantifying Saccharomyces pastorianus, Saccharomyces cerevisiae and Saccharomyces bayanus, which comprises performing a nucleic acid amplification reaction on a nucleic acid sample by the LAMP method, and at the same time, measuring a time required from the initiation of the nucleic acid amplification reaction until a sample solution reaches a certain turbidity and obtaining the number of cells contained in the sample from the measured time. The quantification method of the present invention is specifically as follows.

In the first embodiment according to the present invention, there is provided a method for quantifying Saccharomyces pastorianus, which comprises:

(a) performing a nucleic acid amplification reaction on a nucleic acid contained in a sample by a LAMP method using the LAMP primer set of the first embodiment according to the present invention;
(b′) measuring a time required from the initiation of the nucleic acid amplification reaction until a sample solution reaches a certain turbidity; and
(b″) obtaining the number of cells contained in the sample from the measured time.

In the second embodiment according to the present invention, there is preferably provided a method for quantifying Saccharomyces cerevisiae, which comprises:

(c) performing a nucleic acid amplification reaction on a nucleic acid contained in a sample by a LAMP method using the LAMP primer set of the second embodiment according to the present invention;
(d′) measuring a time required from the initiation of the nucleic acid amplification reaction until a sample solution reaches a certain turbidity; and
(d″) obtaining the number of cells contained in the sample from the measured time.

In the third embodiment according to the present invention, there is provided a method for quantifying Saccharomyces bayanus, which comprises:

(e) performing a nucleic acid amplification reaction on a nucleic acid contained in a sample by a LAMP method using the LAMP primer set of the third embodiment according to the present invention;
(f′) measuring a time required from the initiation of the nucleic acid amplification reaction until a sample solution reaches a certain turbidity; and
(f″) obtaining the number of cells contained in the sample from the measured time.

In the quantification method according to the present invention, a calibration curve has previously been produced using the number of cells and a time required until a sample solution reaches a certain turbidity. Thereafter, the number of cells contained in the sample can be obtained from the measured time based on the calibration curve. The calibration curve can be produced, for example, by preparing samples by diluting cells in a stepwise manner, then performing a nucleic acid amplification method on each sample according to the LAMP method, and then plotting a time required from the initiation of the nucleic amplification reaction until the turbidity becomes 0.1 with respect to the logarithm of the colony formation number of cells.

In the detection method and quantification method according to the present invention, a Loop primer(s) (LF and/or LB) may be added to a primer set consisting of FIP, F3, BIP and B3, and a nucleic acid amplification reaction may be then carried out by the LAMP method. Specifically, in the detection method and quantification method of the first embodiment according to the present invention, in the case of a LAMP primer set consisting of polynucleotides having the base sequences of SEQ ID NOS: 1 to 4 or homologous polynucleotides thereof, a polynucleotide having the base sequence of SEQ ID NO: 5 or a homologous polynucleotide thereof may be added and used as a Loop primer. In the detection method and quantification method of the second embodiment according to the present invention, any one or both of a polynucleotide having the base sequence of SEQ ID NO: 11 or a homologous polynucleotide thereof and a polynucleotide having the base sequence of SEQ ID NO: 12 or a homologous polynucleotide thereof may be added and used as a Loop primer(s). In the detection method and quantification method of the third embodiment according to the present invention, a polynucleotide having the base sequence of SEQ ID NO: 17 or a homologous polynucleotide thereof may be added and used as a Loop primer.

The LAMP primer sets according to the present invention may be used singly or in combination, as appropriate. Using the primer sets according to the present invention in combination, it becomes possible to accurately distinguish Saccharomyces pastorianus, Saccharomyces cerevisiae and Saccharomyces bayanus from one another.

Moreover, the LAMP primer sets according to the present invention can be provided in the form of a kit, singly or in combination. Thus, according to the present invention, there is provided a kit for detecting the yeast of genus Saccharomyces, which comprises a primer set selected from the group consisting of the LAMP primer set of the first embodiment; the LAMP primer set of the second embodiment; the LAMP primer set of the third embodiment; and a combination of a part or all of these primer sets.

The kit according to the present invention, which comprises a LAMP primer set, may comprise reagents (for example, Bst DNA polymerase, a reagent mixed solution for reaction) and apparatuses (for example, a tube for reaction), which are necessary for the implementation of the nucleic acid amplification reaction by the LAMP method.

The kit according to the present invention, which comprises a PCR primer set, may comprise reagents (for example, DNA polymerase, purified water) and apparatuses (for example, a tube for reaction), which are necessary for the implementation of the nucleic acid amplification reaction by the PCR method.

The LAMP primer set of the first embodiment according to the present invention targets an Lg-type MET16 gene, and there is a possibility that it may react with Saccharomyces bayanus, which is not uniform in terms of genomic structure. On the other hand, the LAMP primer set of the third embodiment according to the present invention targets the RAD18 homologous gene of Saccharomyces bayanus, which does not exist in Saccharomyces cerevisiae or Saccharomyces pastorianus, and it is able to detect Saccharomyces bayanus with high accuracy. Accordingly, when Saccharomyces pastorianus is required to be more accurately detected, it is preferable to use the LAMP primer set of the first embodiment according to the present invention in combination with a LAMP primer set used in the detection of Saccharomyces bayanus (preferably the LAMP primer set of the third embodiment). In this case, when an amplification reaction is observed in both the LAMP primer set of the first embodiment according to the present invention and the LAMP primer set used in the detection of Saccharomyces bayanus, or when such an amplification reaction is not observed in the LAMP primer set of the first embodiment but is observed in the LAMP primer set used in the detection of Saccharomyces bayanus, it can be determined that Saccharomyces bayanus is present in the sample. When an amplification reaction is observed in the LAMP primer set of the first embodiment according to the present invention and such an amplification reaction is not observed in the LAMP primer set used in the detection of Saccharomyces bayanus, it can be determined that Saccharomyces pastorianus is present in the sample.

Thus, according to the present invention, there is provided a kit for detecting Saccharomyces pastorianus, which comprises the LAMP primer set of the first embodiment according to the present invention in combination with the LAMP primer set used in the detection of Saccharomyces bayanus.

Moreover, according to the present invention, the method for detecting Saccharomyces pastorianus of the first embodiment according to the present invention may further comprise a step of performing a nucleic acid amplification reaction by a LAMP method using the LAMP primer set used in the detection of Saccharomyces bayanus. This step may include a step of performing a nucleic acid amplification reaction on a nucleic acid sample by a LAMP method and a step of detecting the presence or absence of a nucleic acid amplification product.

In the above descriptions, the LAMP primer set used in the detection of Saccharomyces bayanus is preferably the LAMP primer set of the third embodiment according to the present invention.

Furthermore, when Saccharomyces pastorianus is required to be accurately detected using the LAMP primer set of the first embodiment according to the present invention, it is preferable to use the LAMP primer set of the first embodiment according to the present invention in combination with a LAMP primer set capable of detecting Saccharomyces cerevisiae and Saccharomyces pastorianus. The LAMP primer set for Saccharomyces pastorianus of the first embodiment according to the present invention may cross-react with Saccharomyces bayanus, which is not uniform in terms of genomic structure. The LAMP primer set for use in the detection of Saccharomyces cerevisiae and Saccharomyces pastorianus reacts with the genomic sequence of Saccharomyces cerevisiae contained in Saccharomyces pastorianus, but Saccharomyces bayanus does not have such genomic sequence of Saccharomyces cerevisiae. Thus, the LAMP primer set for use in the detection of Saccharomyces cerevisiae and Saccharomyces pastorianus does not react with Saccharomyces bayanus. In this case, when an amplification reaction is observed in both the LAMP primer set for use in the detection of Saccharomyces pastorianus according to the present invention and the LAMP primer set for use in the detection of Saccharomyces cerevisiae and Saccharomyces pastorianus, it can be determined that Saccharomyces pastorianus is present in the sample. When an amplification reaction occurs by the LAMP primer for use in the detection of Saccharomyces pastorianus according to the present invention and such an amplification reaction is not observed in the LAMP primer set for use in the detection of Saccharomyces cerevisiae and Saccharomyces pastorianus, it can be determined that Saccharomyces bayanus is present in the sample.

Thus, according to the present invention, there is provided a kit for detecting Saccharomyces pastorianus, which comprises the LAMP primer set of the first embodiment according to the present invention in combination with the LAMP primer set used in the detection of Saccharomyces cerevisiae and Saccharomyces pastorianus.

Moreover, according to the present invention, the method for detecting Saccharomyces pastorianus of the first embodiment according to the present invention may further comprise a step of performing a nucleic acid amplification reaction by a LAMP method using the LAMP primer set used in the detection of Saccharomyces cerevisiae and Saccharomyces pastorianus. This step may include a step of performing a nucleic acid amplification reaction on a nucleic acid sample by a LAMP method and a step of detecting the presence or absence of a nucleic acid amplification product.

In the above embodiments, the LAMP primer set used in the detection of Saccharomyces cerevisiae and Saccharomyces pastorianus preferably comprises the following polynucleotides:

a polynucleotide (FIP) having the base sequence of SEQ ID NO: 18, or a polynucleotide consisting of at least 10 bases, which hybridizes with a polynucleotide having a sequence complementary to the base sequence;

a polynucleotide (F3) having the base sequence of SEQ ID NO: 19, or a polynucleotide consisting of at least 10 bases, which hybridizes with a polynucleotide having a sequence complementary to the base sequence;

a polynucleotide (BIP) having the base sequence of SEQ ID NO: 20, or a polynucleotide consisting of at least 10 bases, which hybridizes with a polynucleotide having a sequence complementary to the base sequence; and

a polynucleotide (B3) having the base sequence of SEQ ID NO: 21, or a polynucleotide consisting of at least 10 bases, which hybridizes with a polynucleotide having a sequence complementary to the base sequence.

The aforementioned LAMP primer set used in the detection of Saccharomyces cerevisiae and Saccharomyces pastorianus may further comprise, as a Loop primer, a polynucleotide (LB) having the base sequence of SEQ ID NO: 22, or a polynucleotide consisting of at least 10 bases, which hybridizes with a polynucleotide having a sequence complementary to the base sequence.

Among the detection methods according to the present invention, a specific embodiment of the detection method by a PCR method may include a detection method which comprises performing a nucleic acid amplification reaction on a nucleic acid sample by a PCR method and then detecting the presence or absence of a nucleic acid amplification product.

Specifically, there is provided a method for detecting Saccharomyces pastorianus, which comprises:

(g) performing a nucleic acid amplification reaction on a nucleic acid contained in a sample by a PCR method using the PCR primer set of the first embodiment, third embodiment or fourth embodiment according to the present invention; and
(h) detecting the presence or absence of an amplification product,
wherein the generation of the amplification product indicates the presence of Saccharomyces pastorianus.

As a sample subjected to a nucleic acid amplification process by the PCR method, cells contained in the sample may be cultured and a nucleic acid may be then extracted, or such a nucleic acid may be extracted without culture. Preparation of a nucleic acid sample, such as the culture of cells or the extraction of a nucleic acid, will be described later.

In the nucleic acid amplification process by the PCR method, an amplification reaction is performed on a nucleic acid contained in a sample. Such a nucleic acid amplification reaction carried out by the PCR method has been publicly known. Persons skilled in the art could appropriately determine the conditions for the PCR method or a modified method thereof, and could carry out the PCR method.

Among the detection methods according to the present invention, a specific embodiment of a detection method using a probe may include a detection method which comprises performing the hybridization of the probe according to the present invention with a nucleic acid sample and then detecting the presence or absence of a nucleic acid complex.

Specifically, there is provided a method for detecting Saccharomyces pastorianus, which comprises:

(i) allowing the probe of the first embodiment according to the present invention to come into contact with a nucleic acid contained in a sample; and
(j) detecting the presence or absence of a hybridization complex,
wherein the generation of the hybridization complex indicates the presence of Saccharomyces pastorianus.

In such a detection method using probes, the probes can be labeled before use. Examples of a labeling substance include radioactive elements (for example, ³²P and ¹⁴C), fluorescent compounds (for example, FITC), and molecules associated with an enzyme reaction (for example, peroxidase, alkaline phosphatase).

A hybridization complex can be detected by known methods such as Northern hybridization, Southern hybridization, and colony hybridization.

If the nucleic acid amplification reaction is carried out using the primer set according to the present invention, the yeast of genus Saccharomyces, which causes a decrease in the quality of alcoholic beverages or soft drinks, can be accurately identified at a species level. Accordingly, the primer set and kit according to the present invention can be used in the quality control of alcoholic beverages (for example, beer, low-malt beer, wine, fruit wine, Japanese sake) and/or soft drinks (for example, fruit juice drink) and the examination of an environmental sample (for example, raw material water).

Saccharomyces pastorianus, Saccharomyces cerevisiae and Saccharomyces bayanus may be found as yeast species causing quality deterioration in the production process of beer and low-malt beer or in the final products thereof. Therefore, the LAMP primer set and PCR primer set of the first embodiment; the LAMP primer set of the second embodiment; the LAMP primer set and PCR primer set of the third embodiment; the PCR primer set of the fourth embodiment; and a combination of a part or all of these primer sets can be preferably used in the quality control of beer and low-malt beer.

Saccharomyces cerevisiae and Saccharomyces bayanus may be found as yeast species causing quality deterioration in the production process of wine or in the final products thereof. Therefore, the LAMP primer set of the second embodiment; the LAMP primer set of the third embodiment; and a combination thereof can be preferably used in the quality control of wine.

Saccharomyces cerevisiae and Saccharomyces bayanus may be found as yeast species causing quality deterioration in the production process of soft drinks (in particular, fruit juice drink) or in the final products thereof. Therefore, the LAMP primer set of the second embodiment; the LAMP primer set of the third embodiment; and a combination thereof can be preferably used in the quality control of soft drinks (in particular, fruit juice drink).

### Nucleic Acid Amplification Reaction by LAMP Method

The LAMP primer set according to the present invention can be used as primers for a nucleic acid amplification reaction by a LAMP method. The primer set according to the present invention can also be used as primers not only for the nucleic acid amplification reaction by the LAMP method, but also for a nucleic acid amplification reaction by a modified LAMP method. The principle of the LAMP method and a nucleic acid amplification method utilizing it are well known. In order to carry out the nucleic acid amplification reaction by the LAMP method, descriptions disclosed in WO00/28082, and Notomi T. et al., Nucleic Acids Research, 28(12), e63 (2000) can be used as references.

The nucleic acid amplification reaction by the LAMP method can be carried out using a commercially available LAMP method gene amplification reagent kit. The nucleic acid amplification reaction can be carried out, for example, by mixing sample DNA, a primer solution, and reagents included with a commercially available LAMP method gene amplification reagent kit (for example, a Loopamp DNA amplification kit manufactured by Eiken Chemical Co., Ltd.) in accordance with instructions included with the kit, and then retaining the obtained mixture at a certain temperature (60° C. to 65° C.) so as to react it for a certain period of time (in general, 1 hour).

The nucleic acid amplification reaction by the LAMP method can be carried out via the following processes.

(i) A DNA strand complementary to template DNA is synthesized by the action of strand displacement-type DNA polymerase, using the 3′-terminus of the F2 region of FIP as an origin.
(ii) An F3 primer is annealed to a site outside the FIP, and DNA synthesis is extended by the action of the strand displacement-type DNA polymerase, using the 3′-terminus thereof as an origin, while removing the previously synthesized DNA strand from the FIP.
(iii) A double strand is formed by a DNA strand synthesized from the F3 primer and the template DNA.
(iv) The DNA strand previously synthesized from the FIP is removed due to the DNA strand from the F3 primer, so that it becomes single-stranded DNA. However, this DNA strand has complementary regions F1c and F1 on the 5′-terminal side, and it causes self-annealing so as to form a Loop.
(v) BIP is annealed to the DNA strand that has formed a Loop in the process of (iv) above, and complementary DNA is synthesized using the 3′-terminus of the BIP as an origin. In this process, the Loop is removed and extended. Further, a B3 primer is annealed to a site outside the BIP, and DNA synthesis is extended by the action of the strand displacement-type DNA polymerase, using the 3′-terminus thereof as an origin, while removing the previously synthesized DNA strand from the BIP.
(vi) Double-stranded DNA is formed in the process of (v) above.
(vii) Since the DNA strand synthesized from the BIP that has been removed in the process of (v) above has complementary sequences on both termini, it causes self-annealing to form a Loop, so that it has a dumbbell-like structure.
(viii) Using the aforementioned DNA strand having a dumbbell-like structure as an origin, an amplification cycle of desired DNA is carried out via the annealing of the FIP and then the annealing of the BIP.

It would be obvious to those skilled in the art to carry out the nucleic acid amplification reaction by the LAMP method by appropriately modifying the aforementioned processes. The primer set according to the present invention can also be used in such a modified method.

The LAMP primer set according to the present invention brings on the synthesis of a DNA strand at a temperature of approximately 60° C. to approximately 65° C. (for example, 65° C.), as well as annealing. A reaction is carried out for approximately 1 hour via the annealing reaction and the DNA strand synthesis, so that a nucleic acid can be amplified by 10⁹ to 10¹⁰ times.

If the LAMP primer set according to the present invention is allowed to react with a sample nucleic acid under conditions for the nucleic acid amplification reaction by the LAMP method, a target region of a strain as a detection target is amplified. When such an amplification reaction takes place, a reaction solution becomes clouded due to the influence of magnesium pyrophosphate formed as a by-product. Thus, based on the turbidity, the presence or absence of amplification can be determined by visual observation. The presence or absence of amplification may also be determined by optically measuring the turbidity using a turbidity measurement apparatus. Alternatively, agarose gel electrophoresis or the like may be applied to confirm and detect the presence or absence of a DNA fragment.

If nucleic acid amplification is observed, it means that a target base sequence is present, indicating that the strain as a detection target of the primer set is positive (+). To the contrary, if such nucleic acid amplification is not observed, it means that a target base sequence is absent, indicating that the strain as a detection target of the primer set is negative (−).

### Nucleic Acid Amplification Reaction by PCR Method

The PCR primer set according to the present invention is applied to identify the yeast of genus Saccharomyces, using nucleic acid amplification methods such as a PCR method, an RT-PCR method, a real time PCR method or an in situ PCR method.

If nucleic acid amplification is observed, it means that a target base sequence is present, indicating that the strain as a detection target of the primer set is positive (+). To the contrary, if such nucleic acid amplification is not observed, it means that a target base sequence is absent, indicating that the strain as a detection target of the primer set is negative (−).

In the PCR method, a nucleic acid amplification product can be detected in accordance with known methods such as agarose gel electrophoresis. In addition, in the real time PCR method, such a nucleic acid amplification product can be detected over time in an apparatus formed by integrating a thermal cycler with a spectrophotofluorometer, using an intercalator or a fluorescently labeled probe.

### Detection Target Sample and Preparation Thereof

Examples of a sample used as a detection target of the primer set and kit according to the present invention include: alcoholic beverages such as beer, low-malt beer and wine; soft drinks such as cider, lemon soda and carbonated water; environmental samples such as water collected for use as a raw material; and half-finished products collected from the production process of alcoholic beverages, soft drinks, etc.

When these products are used as samples for the LAMP method or the PCR method, operations such as the concentration, separation and culture of cells existing in the sample, the separation of a nucleic acid from the cells, and the concentration of the nucleic acid may be carried out as pre-treatments. Methods for concentration and separation of cells existing in the sample include filtration and centrifugation, and such methods can be selected, as appropriate. In addition, the cells concentrated and separated from the sample may be further cultured, so as to increase the number of the cells. For the culture, an agar solid medium or a liquid medium, which is suitable for the proliferation of the target yeast strain, may be used. Moreover, in order to select the target yeast strain, an agent such as copper sulfate may be added. In order to release a nucleic acid from cells existing in a beverage sample or an environmental sample or from the cultured cells, a method using a commercially available kit or a method of treating cells with an alkali solution and then heating the cells at 100° C. to release a nucleic acid from them can be selected, for example. Furthermore, if it is necessary to further purify a nucleic acid, the nucleic acid may be purified by a phenol/chloroform treatment, ethanol precipitation, centrifugation, etc., and the purified nucleic acid may be finally re-dissolved in a TE buffer or the like, so that it may be used as template DNA in tests (European Brewery Convention: ANALYTICA-MICROBIOLOGICA-EBC, 2^{nd} ed. 2005, Fachverlag Hans Carl, Nuernberg; Rolf et al.: PCR-Clinical diagnostics and research, Springer-Verlag, Berlin, 1992; Yasuji Oshima et al.: Tanpaku kakusan koso (Proteins, Nucleic acids, Enzymes), Vol. 35, 2523-2541, 1990).

The detection of the yeast of genus Saccharomyces using the primer set and kit according to the present invention can be carried out as follows, for example.

First, the yeast of genus Saccharomyces that are considered to exist in a sample are cultured in a suitable medium. Next, DNA is separated from a colony formed on the agar medium, and the LAMP method or the PCR method using the primer set according to the present invention is then applied to the DNA, so as to amplify the specific gene region of the yeast of genus Saccharomyces. The presence of a gene amplification product indicates the presence of a strain as a target of the primer set.

### EXAMPLES

Hereinafter, the present invention will be specifically described in the following examples. However, these examples are not intended to limit the scope of the present invention.

### Example 1

### Detection of Yeast of Genus Saccharomyces

### (a) Genomic DNA Extraction Method

Cells cultured on an agar plate medium were scraped from the medium, and they were then suspended in sterilized distilled water. This suspension was centrifuged (15,000 rpm, 5 minutes), and a supernatant was then discarded. Sterilized distilled water was added to the precipitated cells again, and the mixed solution was then suspended and centrifuged. A supernatant was discarded, and 100 μl of a solution of PrepMan Ultra (manufactured by Applied Biosystems) was then added to the obtained cells. The mixture was heated at 95° C. for 10 minutes. Thereafter, the resultant was centrifuged at 15,000 rpm for 1 minute, and a supernatant was used as a genomic DNA solution. Otherwise, 100 μl of a 0.1 N NaOH solution was added to the washed cells, and the mixture was then heated at 95° C. for 10 minutes. Thereafter, the resultant was neutralized with a 1 M Tris buffer (pH 7.0), and a supernatant was used as a genomic DNA solution.

### (b) Primers for Use in LAMP

Primers used for various types of yeast strains of genus Saccharomyces as described below were chemically synthesized using eGenome Order (http://genome.e-mp.jp/index.html) manufactured by Fujitsu System Solutions Ltd. or by a method equivalent thereto, and they were then dissolved in a TE buffer (pH 8.0), resulting in a concentration of 100 μM. These solutions were mixed so that they had each predetermined concentrations (FIP and BIP primers: 16 μM; F3 and B3 primers: 2 μM; and LF and LB primers: 8 μM), and they were then diluted.

[Primer set for use in detection of bottom-
fermenting yeast (LGM1LB1)]

(SEQ ID NO: 1)

FIP: CCTTCAGTGTTAAAGTCTGTGGGAAATGACTATCCGGGAAATACT
ATATGCC

(SEQ ID NO: 2)

F3: TCACCATCGACATGCTGTC

(SEQ ID NO: 3)

BIP: TCAGCCCCAGAAGCAAACTATCCAAATTCCGCCTCTGAGACG

(SEQ ID NO: 4)

B3: CCATAGGAAATCACCGTACTTCG

(SEQ ID NO: 5)

LB: ATGTTTATAAGCCAGATGGATG
[Primer set for use in detection of
Saccharomyces cerevisiae (SCM1LF2LB1)]

(SEQ ID NO: 7)

FIP: GGCAGAACCTTGTGATTTTCTTCTACCAAAGTGGAACCTGCACAT
CG

(SEQ ID NO: 8)

F3: TGACAAGTACGATTATCTGGCC

(SEQ ID NO: 9)

BIP: ACTGTCGATTATTGAAATAGACGAACTTAATGGAATACTGTTTAA
CCTGCTCGAACG

(SEQ ID NO: 10)

B3: GTTGTATGGTACATTGTTTGCATCT

(SEQ ID NO: 11)

LF: CACTTATATGTAGCTCTTTGTAGGC

(SEQ ID NO: 12)

LB: AAAAATAAATCCATTGATCAATTGG
[Primer set for use in detection of
Saccharomyces bavanus (SBR2LB1)]

(SEQ ID NO: 13)

FIP: TCCCTCATTACCATCTTCATGAATATGCAACTGAAAATAAAAACC
AGTTCGCC

(SEQ ID NO: 14)

F3: CCAGGCTCATAAAGGAAGCAA

(SEQ ID NO: 15)

BIP: CCGCAAGGTGTTCAAGAAACCTTCACCTCTTGTTCTTCTGTGAG

(SEQ ID NO: 16)

B3: CTGCATCTGTTAAATCTTCATTTGG

(SEQ ID NO: 17)

LB: CAAATGGAGGAGGGGCAA
[Primer set for use in detection of Saccharomyces
cerevisiae and Saccharomvces pastorianus
(SCC1LB1)]

(SEQ ID NO: 18)

FIP: CCTCTTCCAGTTCTTGACTCTTTTCCTAAGATGAAAGTGCCGGGA
GA

(SEQ ID NO: 19)

F3: ACGAAGATGAGAAAGAGGCG

(SEQ ID NO: 20)

BIP: TGACAGCAAGGAGAAGAGCACCCCTCGTTGTTTTCCTCCTCA

(SEQ ID NO: 21)

B3: GTGCTGTATGCTCGTTTTCG

(SEQ ID NO: 22)

LB: GAGCAAGGGGACGAAGGTGA

### (c) Preparation of LAMP Amplification Reaction Solution

A Loopamp DNA amplification kit manufactured by Eiken Chemical Co., Ltd. was used as a gene amplification reagent kit for the LAMP method. 2.5 μl of a genomic DNA solution, 2.5 μl of a primer solution, 12.5 μl of a 2-times concentration reaction buffer, 1 μl of Bst DNA polymerase, and 6.5 μl of sterilized water were added to a reaction tube, and a reaction solution in a total amount of 25 μl was thereby prepared.

### (d) LAMP Reaction

A real-time turbidimeter LA-200 manufactured by Teramecs Co., Ltd. or LA-320C manufactured by Eiken Chemical Co., Ltd. was used for a LAMP reaction. A reaction tube was set, and the reaction solution was allowed to react at a constant temperature of 65° C. (bonnet: 75° C.), and a change in turbidity during the reaction was measured every 6 seconds. The reaction solution whose turbidity was increased was defined as positive, and the reaction solution whose turbidity was not increased was defined as negative.

### (e) Evaluation of Specificity of Primers Used for Various Types of Yeast Strains

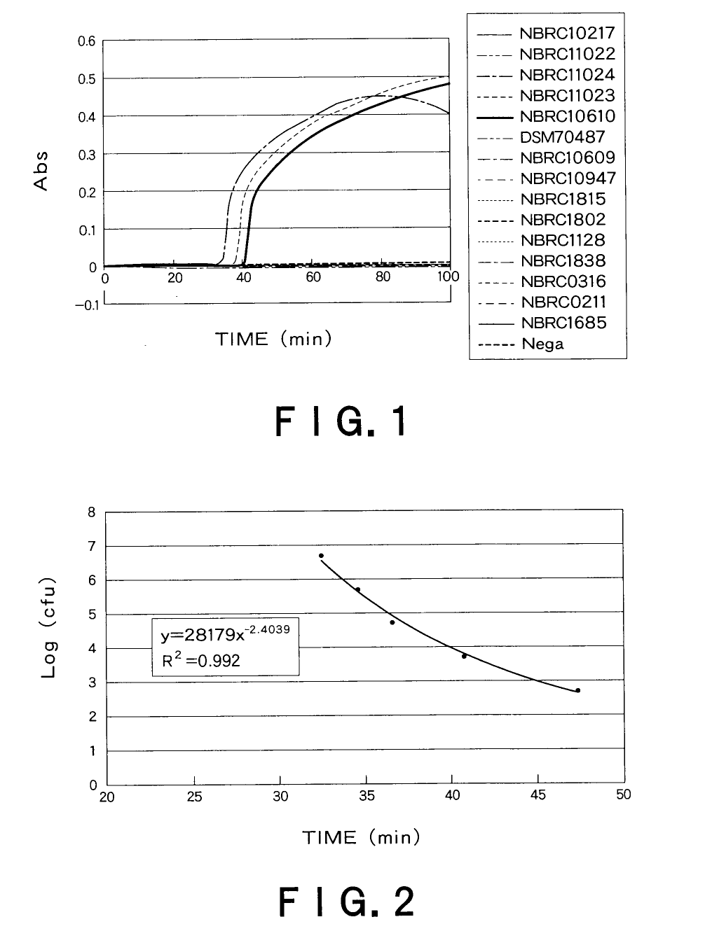

With regard to primers specific for a bottom-fermenting yeast, Saccharomyces cerevisiae and Saccharomyces bayanus, which had been produced for the aforementioned yeast strains, their specificity was evaluated by a LAMP method using standard yeast strains of genus Saccharomyces including each strain of Saccharomyces sensu stricto. As a result, an increase in turbidity was observed with the progress of the DNA amplification within 60 minutes after initiation of the reaction in Saccharomyces pastorianus (the bottom-fermenting yeast) in the case of using LGM1LB1, in Saccharomyces cerevisiae and Saccharomyces cerevisiae var. diastaticus in the case of using SCM1LF2LB1, in Saccharomyces bayanus in the case of using SBR2LB1, and in Saccharomyces cerevisiae and Saccharomyces pastorianus (the bottom-fermenting yeast) in the case of using SCC1LB1 (Table 1). Moreover, when a primer was allowed to react with a strain used as a detection target thereof, an amplification occurred within 60 minutes after initiation of the reaction, and turbidity in the reaction tube was increased (FIG. 1).

TABLE 1
Evaluation of primers using various types or standard
yeast strains of genus Saccharomyces

| Various types of standard yeast | | | | |
|---|---|---|---|---|
| strains of genus Saccharomyces | LGM1LB1 | SCM1LF2LB1 | SBR2LB1 | SCC1LB1 |
| S. cerevisiae NBRC10217 | — | 40 min | — | 20 min |
| S. bayanus NBRC11022 | — | — | 30 min | — |
| S. bayanus NBRC1948 | — | — | 40 min | — |
| S. bayanus NBRC0615 | — | — | 40 min | — |
| S. bayanus NBRC10563 | — | — | 40 min | — |
| S. pastorianus NBRC11024 | 40 min | — | — | 20 min |
| S. pastorianus NBRC11023 | 40 min | — | — | 30 min |
| S. pastorianus NBRC10610 | 40 min | — | — | 30 min |
| S. diastaticus DSM70487 | — | 40 min | — | 20 min |
| S. paradoxus NBRC10609 | — | — | — | — |
| S. paradoxus NBRC0259 | — | — | — | — |
| S. paradoxus NBRC10695 | — | — | — | — |
| S. cariocanus NBRC10947 | — | — | — | — |
| S. mikatae NBRC1815 | — | — | — | — |
| S. kudriavzevii NBRC1802 | — | — | — | — |
| S. exiguus NBRC1128 | — | — | — | — |
| S. servazzii NBRC1838 | — | — | — | — |
| S. unisporus NBRC0316 | — | — | — | — |
| S. dairenensis NBRC0211 | — | — | — | — |
| S. kluyveri NBRC1685 | — | — | — | — |

(the number in each cell of the table: reaction time required until amplification took place; —: no amplification occurred within 80 minutes after the reaction; nt: not tested)

Furthermore, various types of standard yeast strains, yeast strains for use in brewing, wild-type yeast strains isolated at brewery, and wild-type yeast strains isolated from wine were used to evaluate the specificity of various primers such as LGM1LB1, SCM1LF2LB1 and SBR2LB1. As a result, almost no amplification was observed in yeast strains other than those used as detection targets of the aforementioned primers (Tables 2, 3 and 4).

TABLE 2
Evaluation of primers using various types of standard yeast strains

| Various types of standard yeast | | | | |
|---|---|---|---|---|
| strains | LGM1LB1 | SCM1LF2LB1 | SBR2LB1 | SCC1LB1 |
| Pichia anomala NBRC0127 | — | — | — | — |
| Williopsis saturnus NBRC0941 | — | — | — | — |
| Kluyveromyces lactis NBRC1090 | — | — | — | — |
| Candida utilis NBRC0988 | — | — | — | — |
| C. boidinii ATCC48180 | — | — | — | — |
| Zygosaccharomyces bailii | — | — | — | — |
| NBRC1137 | | | | |
| Dekkera anomala DSM70727 | — | — | — | — |
| D. bruxellensis DSM70001 | — | — | — | — |
| D. bruxellensis ATCC64276 | — | — | — | — |
| D. custersiana DSM70736 | — | — | — | — |
| Brettanomyces naardenensis | — | — | — | — |
| NBRC1588 | | | | |

(the number in each cell of the table: reaction time required until amplification took place; —: no amplification occurred within 80 minutes after the reaction)

TABLE 3
Evaluation of primers using various types of yeast strains for
use in brewing and wild-type yeast strains isolated at brewery

| Brewing yeast, wild-type beer | | | | |
|---|---|---|---|---|
| yeast | LGM1LB1 | SCM1LF2LB1 | SBR2LB1 | SCC1LB1 |
| Bottom-fermenting yeast BFY61 | 40 min | — | — | 20 min |
| Bottom-fermenting yeast BFY70 | 40 min | — | — | 20 min |
| Bottom-fermenting yeast BFY84 | 40 min | — | — | 20 min |
| Bottom-fermenting yeast BFY448 | 50 min | — | — | 20 min |
| Top-fermenting yeast TFY3 | — | 40 min | — | 20 min |
| Top-fermenting yeast TFY23 | — | 40 min | — | 20 min |
| Trichosporon cutaneum WY54 | — | — | — | — |
| C. intermedia WY55-1 | — | — | — | — |
| Debaryomyces hansenii WY69 | — | — | — | — |
| P. membranifaciens WY75 | — | — | — | — |
| Rhodotorula graminis WY93 | — | — | — | — |
| D. bruxellensis WY97 | — | — | — | — |
| S. cerevisiae WY101 | — | 40 min | — | 20 min |
| S. diastaticus WY126 | — | 50 min | — | 20 min |

(the number in each cell of the table: reaction time required until amplification took place; —: no amplification occurred within 80 minutes after the reaction)

TABLE 4
Evaluation of primers using wild-type yeast strains isolated from various types of wines

| Wild-type wine yeast | LGM1LB1 | SCM1LF2LB1 | SBR2LB1 | SCC1LB1 |
|---|---|---|---|---|
| Z. bailii WLY9 | — | — | — | — |
| S. cerevisiae WLY10 | — | 40 min | — | 20 min |
| P. membranifaciens WLY13 | — | — | — | — |
| Lodderomyces elongisporus | — | — | — | — |
| WLY14 | | | | |
| Aureobasidium pullulans WLY15 | — | — | — | — |
| Rhodosporidium fluviale WLY16 | — | — | — | — |
| P. anomala WLY17 | — | — | — | — |
| P. guilliermondii WLY18 | — | — | — | — |

(the number in each cell of the table: reaction time required until amplification took place; —: no amplification occurred within 80 minutes after the reaction)

The above results demonstrated that the primer sets according to the present invention produced for various types of yeast strains of genus Saccharomyces can accurately detect the yeasts of genus Saccharomyces as detection targets at a species level. According to the previous reports, gene fragments have been amplified from yeasts using the same primers, and thereafter, a difference in base sequences has been analyzed using a restriction enzyme cleavage pattern, DGGE, etc., in many cases. Using the primers according to the present invention, however, the 3 types of yeast strains belonging to genus Saccharomyces can be identified and detected only by confirming the presence or absence of gene amplification.

### Example 2

### Detection Limit of LAMP Method

In order to analyze the amplification efficiency of the LAMP method, the cells of a bottom-fermenting yeast (BFY70, Saccharomyces pastorianus), Saccharomyces cerevisiae NBRC10217 and Saccharomyces bayanus NBRC1948, which had been cultured on an agar plate medium, were diluted with sterilized water in a stepwise manner, and DNA was then extracted by the aforementioned method. The extracted DNA was subjected to the LAMP method. As a result, in the case of the LAMP method, amplification was observed even from small quantities of cells such as a level of 10² to 10³ cfu.

In addition, when genomic DNA extracted from the diluted solution of the cells at each dilution step was used, the time at which the turbidity exceeded 0.1 in the LAMP reaction was defined as a detection time, and a graph was formed from a logarithm of the detection time of each primer and the number of colonies formed. As a result of exponential approximation, an approximate curve of high correlation coefficient could be formed (R²=0.98-0.99). The approximate curve of LGM1LB1 is as shown in FIG. 2. Moreover, the relationship between the detection limit of each primer and the correlation coefficient of a calibration curve is as described below. It was demonstrated that, by forming such a calibration curve, the presence of the yeast of genus Saccharomyces contained in a sample can be quantitatively measured within the range from 10² to 10⁷ cfu or from 10³ to 10⁷ cfu.

[Detection limit of each primer and correlation
coefficient of calibration curve]

| | | Calibration curve |
|---|---|---|
| | Calibration curve | correlation |
| Detection limit | approximate expression | coefficient (R²) |
| LGM1LB1 | 5.2 × 10² cfu y = 28179x^{-2.4039} | 0.992 |
| SCM1LF2LB1 | 4.4 × 10³ cfu y = 6875x^{-1.8959} | 0.981 |
| SBR2LB1 | 1.9 × 10² cfu y = 1431.3x^{-1.8361} | 0.982 |

### Example 3

### Detection of Cells in Wine and Beer

In the production of wine, in general, a large amount of yeast of genus Saccharomyces used as a wine yeast is added to fruit juice for fermentation. The number of yeast cells that contaminate the fruit juice from outside is significantly smaller than that of the yeast of genus Saccharomyces. Thus, a large amount of Saccharomyces cerevisiae was suspended in wine, and thereafter, the cell diluted solution of Saccharomyces bayanus NBRC1948 as prepared by diluting with wine was mixed therewith, followed by cell collection and washing. Thereafter, the cells were gathered, and DNA was then extracted therefrom. Using SBR2LB1 as a primer set, a LAMP method was carried out, and thus, the possibility of detection of Saccharomyces bayanus was analyzed. As a result, it was found that, regardless of the inhibition of the reaction by wine and the presence of Saccharomyces cerevisiae, Saccharomyces bayanus can be detected at almost the same detection limit as in the case of suspending it in sterilized water. Moreover, even in a case where a bottom-fermenting yeast was suspended in beer and where the cell diluted solution of Saccharomyces bayanus was mixed therewith, the same results were obtained.

Detection limit of Saccharomyces bayanus Wine+Saccharomyces cerevisiae (5×10⁷ cells) 3.9×10² cfu Beer+bottom-fermenting yeast (1×10⁷ cells) 3.2×10² cfu

### Example 4

### Evaluation of Primers Described in Patent Publication

### (a) Primers Described in Patent Publication

A primer set for use in the detection of genus Saccharomyces (SSC1LB1) and a primer set for use in the detection of a bottom-fermenting yeast (SBFY1LF1LB1), as described below, which had been described in the patent publication of Tsuchiya et al. (WO2005/093059), were used to prepare primer solutions used for the LAMP method in the same manner as in Example 1(b). The primer set for use in the detection of genus Saccharomyces (SSC1LB1) targets the D2 region of an rRNA gene, whereas the primer set for use in the detection of the bottom-fermenting yeast (SBFY1LF1LB1) targets a melibiase gene.

[Primer set for use in the detection of genus
Saccharomyces (SSC1LB1)]

(SEQ ID NO: 31)

FIP: TGCGAGATTCCCCTACCCCAGACATGGTGTTTTGTGCC

(SEQ ID NO: 32)

F3: AGACCGATAGCGAACAAGTA

(SEQ ID NO: 33)

BIP: CTGTGGGAATACTGCCAGCTGGCCGTGTTTCAAGACGGGCGG

(SEQ ID NO: 34)

B3: CTTGGTCCGTGTTTCAAGAC

(SEQ ID NO: 35)

LB: CAAGGATGCTGGCATAATGGTT
[Primer set for use in the detection of the
bottom-fermenting yeast (SBFY1LF1LB1)]

(SEQ ID NO: 36)

FIP: GCAATTGCTCACTGACGTCGCACACCCCTCAAATGGGTTGG

(SEQ ID NO: 37)

F3: CCGAGTTACAATGGCCTTGG

(SEQ ID NO: 38)

BIP: ACCGCTGACCGGATTTCTGAAAACTAGACCAGCAGTCATCCA

(SEQ ID NO: 39)

B3: CCTTGTCTGCAACGAGTGT

(SEQ ID NO: 40)

LF: GGCAAATGTGTTCCAATTGTC

(SEQ ID NO: 41)
LB: GGACTAAAGGATTTGGGTTACAC

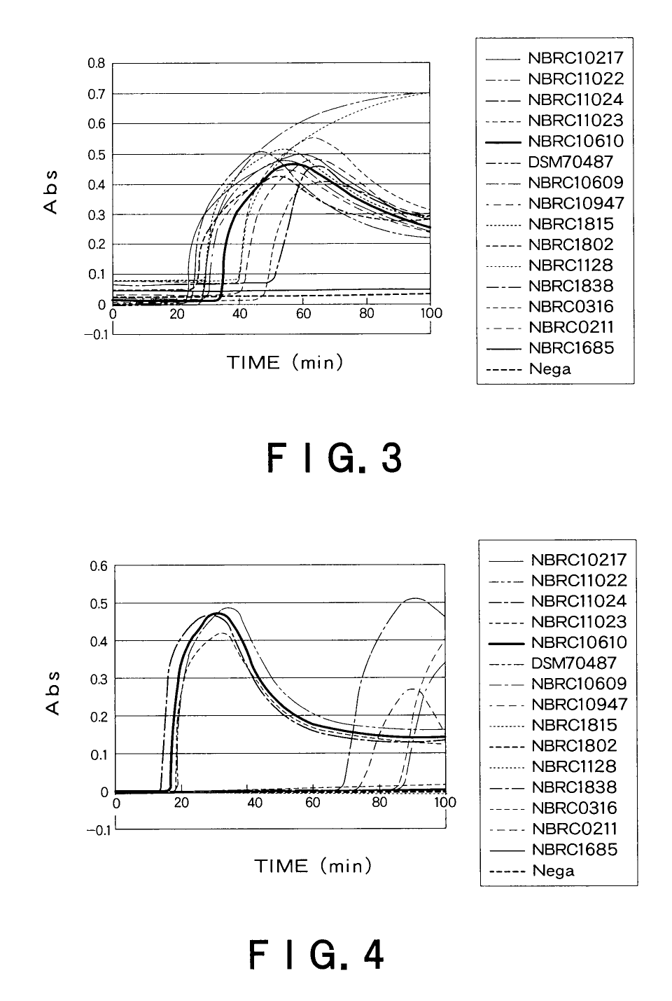

In accordance with the descriptions of Example 1(c) and (d), using the aforementioned primer sets, various types of strains were detected by the LAMP method. The results are as shown in FIGS. 3 and 4.

### (b) Evaluation of Primers

As a result, it was found that SSC1LB1 reacted with almost all the examined cell strains of yeasts of genus Saccharomyces, and thus that it cannot be used in the identification of the yeasts of genus Saccharomyces. In addition, SBFY1LF1LB1 reacted with Saccharomyces bayanus as well as with the bottom-fermenting yeast. Such SBFY1LF1LB1 had been designed from the melibiase gene of the bottom-fermenting yeast. Since a base sequence showing 96% homology with the genome of Saccharomyces bayanus was present in the region used in the designing the primer set, it was considered that a cross-reaction took place.

### Example 3

### Evaluation of PCR Primers Using Sc-Type-Lg-Type Chromosomal Translocation Regions

As stated above, the Sc-type chromosome XVI of a bottom-fermenting yeast was translocated with an Lg-type chromosome within a region from the ORF of the right arm GPH1 to the ORF of QCR2. The Lg-type chromosome III of such a bottom-fermenting yeast was translocated with an Sc-type chromosome from the MAT locus of the right arm to the terminus thereof. Moreover, the Lg-type chromosome VII of the bottom-fermenting yeast was translocated with an Sc-type chromosome from the KEM1 gene ORF of the left arm to the terminus thereof. Primers used for the PCR method were designed from the base sequence on the side of the Sc-type chromosome and the base sequence on the side of the Lg-type chromosome, such that the chromosomal translocation positions of the chromosome III, chromosome VII and chromosome XVI of the bottom-fermenting yeast could be flanked with them. Using various types of yeasts of genus Saccharomyces, the primers were evaluated.

Ex Taq manufactured by Takara Bio Inc. was used for the PCR method. The base sequences of primers used for the PCR are as follows.

[Primer set used for chromosome III translocation
region (amplification product: approximately
3.2 kb)]

(SEQ ID NO: 23)

IIIjunc1 (Lg-type side): TGTTGGGGTGTACTATGGTCTTT

(SEQ ID NO: 24)

IIIjunc2 (Sc-type side): ACAAAGAATGATGCTAAGAATTGA
[Primer set used for chromosome VII translocation
region (amplification product: approximately
350 bp)]

(SEQ ID NO: 25)

VIISL1 (Lg-type side): CGACTCAAACTGTATTACTCC

(SEQ ID NO: 26)

VIISL2 (Sc-type side): AATTTTGATGTTCAAGCG
[Primer set used for chromosome XVI translocation
region (amplification product: XVISL1-2
approximately 720 bp; XVISL3-4 approximately
630 bp)]

(SEQ ID NO: 27)

XVISL1 (Lg-type side): CGACAGAGTTGACCAGTTTG

(SEQ ID NO: 28)

XVISL2 (Sc-type side): GTTCTTCTTGCAAGATGTGG

(SEQ ID NO: 29)

XVISL3 (Lg-type side): CCTTGGCAGATGTGTTGTAT

(SEQ ID NO: 30)

XVISL4 (Sc-type side): CTTGCCCTTCTTCAAATCCG

These reagents were mixed with one another in accordance with the instruction of the Ex Taq manufactured by Takara Bio Inc. (total amount: 15 μl), and the mixture was then set in a thermal cycler. A temperature program of 94° C.-30 seconds, 55° C.-30 seconds, and 72° C.-1 minute (in the case of the primer used for the chromosome III translocation region, 72° C.-2 minutes) was repeated 30 times, so as to carry out a PCR reaction. As a result, a band with a putative molecular weight appeared, when each PCR primer set was used for the bottom-fermenting yeast. The presence or absence of an amplification product is as shown in Table 5.

TABLE 5
Evaluation of primers using various types of yeast strains of genus Saccharomyces

| | IIIjunc1-2 | VIISL1-2 | XVISL1-2 | XVISL3-4 |
|---|---|---|---|---|
| Bottom-fermenting yeast BFY70 | + | + | + | + |
| Bottom-fermenting yeast BFY84 | + | + | + | + |
| Bottom-fermenting yeast BFY85 | + | + | + | + |
| Bottom-fermenting yeast W34/70 | + | + | + | + |
| Bottom-fermenting yeast BFY427 | + | + | + | + |
| Bottom-fermenting yeast BFY253 | + | + | + | + |
| S. cerevisiae S288C | − | − | − | − |
| S. bayanus NBRC11022 | − | − | − | − |

(+: presence of amplification; −: absence of amplification)

1. A probe or primer for use in the detection of Saccharomyces pastorianus, which consists of a polynucleotide consisting of at least 10 bases, which hybridizes with a polynucleotide having the base sequence of SEQ ID NO: 6, or a polynucleotide consisting of at least 10 bases, which hybridizes with a polynucleotide having a sequence complementary to the base sequence of SEQ ID NO: 6.

2. A LAMP primer set for use in the detection of Saccharomyces pastorianus, which consists of two or more types of the primers according to claim 1.

3. The LAMP primer set according to claim 2, which comprises the following polynucleotides:
a polynucleotide (FIP) having the base sequence of SEQ ID NO: 1, or a polynucleotide consisting of at least 10 bases, which hybridizes with a polynucleotide having a sequence complementary to the base sequence;
a polynucleotide (F3) having the base sequence of SEQ ID NO: 2, or a polynucleotide consisting of at least 10 bases, which hybridizes with a polynucleotide having a sequence complementary to the base sequence;
a polynucleotide (BIP) having the base sequence of SEQ ID NO: 3, or a polynucleotide consisting of at least 10 bases, which hybridizes with a polynucleotide having a sequence complementary to the base sequence; and
a polynucleotide (B3) having the base sequence of SEQ ID NO: 4, or a polynucleotide consisting of at least 10 bases, which hybridizes with a polynucleotide having a sequence complementary to the base sequence.

4. The LAMP primer set according to claim 3, which further comprises a polynucleotide (LB) having the base sequence of SEQ ID NO: 5, or a polynucleotide consisting of at least 10 bases, which hybridizes with a polynucleotide having a sequence complementary to the base sequence.

5. A PCR primer set for use in the detection of Saccharomyces pastorianus, which consists of two or more types of the primers according to claim 1.

6. A LAMP primer set for use in the detection of Saccharomyces cerevisiae, which comprises the following polynucleotides:
a polynucleotide (FIP) having the base sequence of SEQ ID NO: 7, or a polynucleotide consisting of at least 10 bases, which hybridizes with a polynucleotide having a sequence complementary to the base sequence;
a polynucleotide (F3) having the base sequence of SEQ ID NO: 8, or a polynucleotide consisting of at least 10 bases, which hybridizes with a polynucleotide having a sequence complementary to the base sequence;
a polynucleotide (BIP) having the base sequence of SEQ ID NO: 9, or a polynucleotide consisting of at least 10 bases, which hybridizes with a polynucleotide having a sequence complementary to the base sequence; and
a polynucleotide (B3) having the base sequence of SEQ ID NO: 10, or a polynucleotide consisting of at least 10 bases, which hybridizes with a polynucleotide having a sequence complementary to the base sequence.

7. The LAMP primer set according to claim 6, which further comprises any one or both of the following polynucleotides:
a polynucleotide (LF) having the base sequence of SEQ ID NO: 11, or a polynucleotide consisting of at least 10 bases, which hybridizes with a polynucleotide having a sequence complementary to the base sequence; and
a polynucleotide (LB) having the base sequence of SEQ ID NO: 12, or a polynucleotide consisting of at least 10 bases, which hybridizes with a polynucleotide having a sequence complementary to the base sequence.

8. A LAMP primer set for use in the detection of Saccharomyces bayanus, which comprises the following polynucleotides:
a polynucleotide (FIP) having the base sequence of SEQ ID NO: 13, or a polynucleotide consisting of at least 10 bases, which hybridizes with a polynucleotide having a sequence complementary to the base sequence;
a polynucleotide (F3) having the base sequence of SEQ ID NO: 14, or a polynucleotide consisting of at least 10 bases, which hybridizes with a polynucleotide having a sequence complementary to the base sequence;
a polynucleotide (BIP) having the base sequence of SEQ ID NO: 15, or a polynucleotide consisting of at least 10 bases, which hybridizes with a polynucleotide having a sequence complementary to the base sequence; and
a polynucleotide (B3) having the base sequence of SEQ ID NO: 16, or a polynucleotide consisting of at least 10 bases, which hybridizes with a polynucleotide having a sequence complementary to the base sequence.

9. The primer set according to claim 8, which further comprises a polynucleotide (LB) having the base sequence of SEQ ID NO: 17, or a polynucleotide consisting of at least 10 bases, which hybridizes with a polynucleotide having a sequence complementary to the base sequence.

10. A PCR primer set for use in the detection of Saccharomyces pastorianus, which comprises the following polynucleotides:
a polynucleotide having the base sequence of SEQ ID NO: 23, or a polynucleotide consisting of at least 10 bases, which hybridizes with a polynucleotide having a sequence complementary to the base sequence; and
a polynucleotide having the base sequence of SEQ ID NO: 24, or a polynucleotide consisting of at least 10 bases, which hybridizes with a polynucleotide having a sequence complementary to the base sequence.

11. A PCR primer set for use in the detection of Saccharomyces pastorianus, which comprises the following polynucleotides:
a polynucleotide having the base sequence of SEQ ID NO: 25, or a polynucleotide consisting of at least 10 bases, which hybridizes with a polynucleotide having a sequence complementary to the base sequence; and
a polynucleotide having the base sequence of SEQ ID NO: 26, or a polynucleotide consisting of at least 10 bases, which hybridizes with a polynucleotide having a sequence complementary to the base sequence.

12. A PCR primer set for use in the detection of Saccharomyces pastorianus, which comprises the following polynucleotides:
a polynucleotide having the base sequence of SEQ ID NO: 27, or a polynucleotide consisting of at least 10 bases, which hybridizes with a polynucleotide having a sequence complementary to the base sequence; and
a polynucleotide having the base sequence of SEQ ID NO: 28, or a polynucleotide consisting of at least 10 bases, which hybridizes with a polynucleotide having a sequence complementary to the base sequence.

13. A PCR primer set for use in the detection of Saccharomyces pastorianus, which comprises the following polynucleotides:
a polynucleotide having the base sequence of SEQ ID NO: 29, or a polynucleotide consisting of at least 10 bases, which hybridizes with a polynucleotide having a sequence complementary to the base sequence; and
a polynucleotide having the base sequence of SEQ ID NO: 30, or a polynucleotide consisting of at least 10 bases, which hybridizes with a polynucleotide having a sequence complementary to the base sequence.

14. The probe or primer according to claim 1, wherein the polynucleotide consisting of at least 10 bases hybridizing with the polynucleotide having the base sequence of SEQ ID NO: 6, and the polynucleotide consisting of at least 10 bases hybridizing with the polynucleotide having a sequence complementary to the base sequence of SEQ ID NO: 6 comprise at least 10 contiguous nucleotides of a sequence complementary to the base sequence of SEQ ID NO: 6, and at least 10 contiguous nucleotides of the base sequence of SEQ ID NO: 6, respectively.

15. The probe or primer according to claim 1, wherein the polynucleotide consisting of at least 10 bases hybridizing with the polynucleotide having the base sequence of SEQ ID NO: 6, and the polynucleotide consisting of at least 10 bases hybridizing with the polynucleotide having a sequence complementary to the base sequence of SEQ ID NO: 6 are a polynucleotide having at least 90% identity with a sequence complementary to the base sequence of SEQ ID NO: 6, and a polynucleotide having at least 90% identity with the base sequence of SEQ ID NO: 6, respectively.

16. The probe or primer according to claim 1, wherein the polynucleotide consisting of at least 10 bases hybridizing with the polynucleotide having the base sequence of SEQ ID NO: 6, and the polynucleotide consisting of at least 10 bases hybridizing with the polynucleotide having a sequence complementary to the base sequence of SEQ ID NO: 6 are a polynucleotide which consists of a modified base sequence in which one or several nucleotides are modified in a sequence complementary to the base sequence of SEQ ID NO: 6 and hybridizes with the polynucleotide having the base sequence of SEQ ID NO: 6, and a polynucleotide which consists of a modified base sequence in which one or several nucleotides are modified in the base sequence of SEQ ID NO: 6 and hybridizes with the polynucleotide having a sequence complementary to the base sequence of SEQ ID NO: 6, respectively.

17. The primer set according to claim 3, wherein the polynucleotide consisting of at least 10 bases hybridizing with a polynucleotide having a sequence complementary to the base sequence of SEQ ID NO: 1 to 5, 7 to 17, or 23 to 30 comprises at least 10 contiguous nucleotides of the corresponding base sequence.

18. The primer set according to claim 3, wherein the polynucleotide consisting of at least 10 bases hybridizing with a polynucleotide having a sequence complementary to the base sequence of SEQ ID NO: 1 to 4 a polynucleotide having at least 90% identity with the corresponding base sequence.

19. The primer set according to claim 3, wherein the polynucleotide consisting of at least 10 bases hybridizing with a polynucleotide having a sequence complementary to the base sequence of SEQ ID NO: 1 to 4 is a polynucleotide which consists of a modified base sequence in which one or several nucleotides are modified in the corresponding base sequence and hybridizes with a polynucleotide having a sequence complementary to the corresponding base sequence.

20. A kit for detecting Saccharomyces pastorianus, which comprises the primer set according to claim 2 in combination with a LAMP primer set for use in the detection of Saccharomyces bayanus.

21. The kit according to claim 20, wherein the LAMP primer set for use in the detection of Saccharomyces bayanus is the LAMP primer set according to claim 8.

22. A kit for detecting Saccharomyces pastorianus, which comprises the LAMP primer set according to claim 2 in combination with a LAMP primer set for use in the detection of Saccharomyces cerevisiae and Saccharomyces pastorianus.

23. The detection kit according to claim 22, wherein the LAMP primer set for use in the detection of Saccharomyces cerevisiae and Saccharomyces pastorianus comprises the following polynucleotides:
a polynucleotide (FIP) having the base sequence of SEQ ID NO: 18, or a polynucleotide consisting of at least 10 bases, which hybridizes with a polynucleotide having a sequence complementary to the base sequence;
a polynucleotide (F3) having the base sequence of SEQ ID NO: 19, or a polynucleotide consisting of at least 10 bases, which hybridizes with a polynucleotide having a sequence complementary to the base sequence;
a polynucleotide (BIP) having the base sequence of SEQ ID NO: 20, or a polynucleotide consisting of at least 10 bases, which hybridizes with a polynucleotide having a sequence complementary to the base sequence; and
a polynucleotide (B3) having the base sequence of SEQ ID NO: 21, or a polynucleotide consisting of at least 10 bases, which hybridizes with a polynucleotide having a sequence complementary to the base sequence.

24. The detection kit according to claim 23, which further comprises a polynucleotide (LB) having the base sequence of SEQ ID NO: 22, or a polynucleotide consisting of at least 10 bases, which hybridizes with a polynucleotide having a sequence complementary to the base sequence, as a LAMP primer for use in the detection of Saccharomyces cerevisiae and Saccharomyces pastorianus.

25-34. (canceled)


SEQUENCE LISTING (41 sequences)

SEQ ID NO: 1 - DNA, 52 bp, Artificial Sequence
  Description of Artificial Sequence Synthetic primer
ccttcagtgttaaagtctgtgggaaatgactatccgggaaatactatatgcc

SEQ ID NO: 2 - DNA, 19 bp, Artificial Sequence
  Description of Artificial Sequence Synthetic primer
tcaccatcgacatgctgtc

SEQ ID NO: 3 - DNA, 42 bp, Artificial Sequence
  Description of Artificial Sequence Synthetic primer
tcagccccagaagcaaactatccaaattccgcctctgagacg

SEQ ID NO: 4 - DNA, 23 bp, Artificial Sequence
  Description of Artificial Sequence Synthetic primer
ccataggaaatcaccgtacttcg

SEQ ID NO: 5 - DNA, 22 bp, Artificial Sequence
  Description of Artificial Sequence Synthetic primer
atgtttataagccagatggatg

SEQ ID NO: 6 - DNA, 785 bp, Saccharomyces pastorianus
atgaaaagttaccatttgaacaatgacattattgtcacacaggaacagttagatcattggaacgaacaattagccaagttggaaacggcacaagagattatcgaatggtcgctagtggctttcccacaccttttccaaaccactgcgtttggactgacaggtttggtcaccatcgacatgctgtcgaaactatccgggaaatactatatgccggaattattatttatagacactttgcaccatttcccacagactttaacactgaaggatacaatcgagcaaaagtattatcagccccagaagcaaactatccatgtttataagccagatggatgcgtctcagaggcggaatttgcctcgaagtacggtgatttcctatgggagaaagacgacgataagtacgactatttggctaaagtagaacctgcgcaccgtgcttacaaagaactgcgcgtaagtgccgtcttcacaggcagaagaaagtcgcagggttctgctcgctctcagttgtcgcttattgaaattgacgagcttaacgggatcttgaagataaatccattgattaattggacattcgagcaagttaaacaatatatagatgcaaacaatgtaccatacaatgagcttttggatctcgggtatagatccgttggtgattaccattccacacaacctgtaaaggaaggtgaagatgaaagagcaggaaggtggaagggcaagacaaagactgagtgtggaattcacgaagccagtagattcgcccaatttctaaaacaagacgccta

SEQ ID NO: 7 - DNA, 47 bp, Artificial Sequence
  Description of Artificial Sequence Synthetic primer
ggcagaaccttgtgattttcttctaccaaagtggaacctgcacatcg

SEQ ID NO: 8 - DNA, 22 bp, Artificial Sequence
  Description of Artificial Sequence Synthetic primer
tgacaagtacgattatctggcc

SEQ ID NO: 9 - DNA, 57 bp, Artificial Sequence
  Description of Artificial Sequence Synthetic primer
actgtcgattattgaaatagacgaacttaatggaatactgtttaacctgctcgaacg

SEQ ID NO: 10 - DNA, 25 bp, Artificial Sequence
  Description of Artificial Sequence Synthetic primer
gttgtatggtacattgtttgcatct

SEQ ID NO: 11 - DNA, 25 bp, Artificial Sequence
  Description of Artificial Sequence Synthetic primer
cacttatatgtagctctttgtaggc

SEQ ID NO: 12 - DNA, 25 bp, Artificial Sequence
  Description of Artificial Sequence Synthetic primer
aaaaataaatccattgatcaattgg

SEQ ID NO: 13 - DNA, 53 bp, Artificial Sequence
  Description of Artificial Sequence Synthetic primer
tccctcattaccatcttcatgaatatgcaactgaaaataaaaaccagttcgcc

SEQ ID NO: 14 - DNA, 21 bp, Artificial Sequence
  Description of Artificial Sequence Synthetic primer
ccaggctcataaaggaagcaa

SEQ ID NO: 15 - DNA, 44 bp, Artificial Sequence
  Description of Artificial Sequence Synthetic primer
ccgcaaggtgttcaagaaaccttcacctcttgttcttctgtgag

SEQ ID NO: 16 - DNA, 25 bp, Artificial Sequence
  Description of Artificial Sequence Synthetic primer
ctgcatctgttaaatcttcatttgg

SEQ ID NO: 17 - DNA, 18 bp, Artificial Sequence
  Description of Artificial Sequence Synthetic primer
caaatggaggaggggcaa

SEQ ID NO: 18 - DNA, 47 bp, Artificial Sequence
  Description of Artificial Sequence Synthetic primer
cctcttccagttcttgactcttttcctaagatgaaagtgccgggaga

SEQ ID NO: 19 - DNA, 20 bp, Artificial Sequence
  Description of Artificial Sequence Synthetic primer
acgaagatgagaaagaggcg

SEQ ID NO: 20 - DNA, 42 bp, Artificial Sequence
  Description of Artificial Sequence Synthetic primer
tgacagcaaggagaagagcacccctcgttgttttcctcctca

SEQ ID NO: 21 - DNA, 20 bp, Artificial Sequence
  Description of Artificial Sequence Synthetic primer
gtgctgtatgctcgttttcg

SEQ ID NO: 22 - DNA, 20 bp, Artificial Sequence
  Description of Artificial Sequence Synthetic primer
gagcaaggggacgaaggtga

SEQ ID NO: 23 - DNA, 23 bp, Artificial Sequence
  Description of Artificial Sequence Synthetic primer
tgttggggtgtactatggtcttt

SEQ ID NO: 24 - DNA, 24 bp, Artificial Sequence
  Description of Artificial Sequence Synthetic primer
acaaagaatgatgctaagaattga

SEQ ID NO: 25 - DNA, 21 bp, Artificial Sequence
  Description of Artificial Sequence Synthetic primer
cgactcaaactgtattactcc

SEQ ID NO: 26 - DNA, 18 bp, Artificial Sequence
  Description of Artificial Sequence Synthetic primer
aattttgatgttcaagcg

SEQ ID NO: 27 - DNA, 20 bp, Artificial Sequence
  Description of Artificial Sequence Synthetic primer
cgacagagttgaccagtttg

SEQ ID NO: 28 - DNA, 20 bp, Artificial Sequence
  Description of Artificial Sequence Synthetic primer
gttcttcttgcaagatgtgg

SEQ ID NO: 29 - DNA, 20 bp, Artificial Sequence
  Description of Artificial Sequence Synthetic primer
ccttggcagatgtgttgtat

SEQ ID NO: 30 - DNA, 20 bp, Artificial Sequence
  Description of Artificial Sequence Synthetic primer
cttgcccttcttcaaatccg

SEQ ID NO: 31 - DNA, 38 bp, Artificial Sequence
  Description of Artificial Sequence Synthetic primer
tgcgagattcccctaccccagacatggtgttttgtgcc

SEQ ID NO: 32 - DNA, 20 bp, Artificial Sequence
  Description of Artificial Sequence Synthetic primer
agaccgatagcgaacaagta

SEQ ID NO: 33 - DNA, 42 bp, Artificial Sequence
  Description of Artificial Sequence Synthetic primer
ctgtgggaatactgccagctggccgtgtttcaagacgggcgg

SEQ ID NO: 34 - DNA, 20 bp, Artificial Sequence
  Description of Artificial Sequence Synthetic primer
cttggtccgtgtttcaagac

SEQ ID NO: 35 - DNA, 22 bp, Artificial Sequence
  Description of Artificial Sequence Synthetic primer
caaggatgctggcataatggtt

SEQ ID NO: 36 - DNA, 41 bp, Artificial Sequence
  Description of Artificial Sequence Synthetic primer
gcaattgctcactgacgtcgcacacccctcaaatgggttgg

SEQ ID NO: 37 - DNA, 20 bp, Artificial Sequence
  Description of Artificial Sequence Synthetic primer
ccgagttacaatggccttgg

SEQ ID NO: 38 - DNA, 42 bp, Artificial Sequence
  Description of Artificial Sequence Synthetic primer
accgctgaccggatttctgaaaactagaccagcagtcatcca

SEQ ID NO: 39 - DNA, 19 bp, Artificial Sequence
  Description of Artificial Sequence Synthetic primer
ccttgtctgcaacgagtgt

SEQ ID NO: 40 - DNA, 21 bp, Artificial Sequence
  Description of Artificial Sequence Synthetic primer
ggcaaatgtgttccaattgtc

SEQ ID NO: 41 - DNA, 23 bp, Artificial Sequence
  Description of Artificial Sequence Synthetic primer
ggactaaaggatttgggttacac